(19) United States
(12) Patent Application Publication
(10) Pub. No.: US 2025/0360212 A1
(43) Pub. Date: Nov. 27, 2025

(54) COMPOSITIONS AND USES OF PSCA TARGETED CHIMERIC ANTIGEN RECEPTOR MODIFIED CELLS EXPRESSING IL-15

(71) Applicant: City of Hope, Duarte, CA (US)

(72) Inventors: Jianhua Yu, Duarte, CA (US); Michael A. Caligiuri, Duarte, CA (US); Zhenyu Dai, Duarte, CA (US)

(21) Appl. No.: 18/981,030

(22) Filed: Dec. 13, 2024

Related U.S. Application Data
(60) Provisional application No. 63/610,651, filed on Dec. 15, 2023.

(51) Int. Cl. A61K 40/42; A61K 40/15; A61K 40/31; A61K 40/35; A61P 35/00; C07K 14/54; C07K 14/715; C07K 14/725; C07K 16/30; C12N 5/0783

(52) U.S. Cl. CPC A61K 40/4274; A61K 40/15; A61K 40/31; A61K 40/35; A61P 35/00; C07K 14/5443; C07K 14/7051; C07K 14/7155; C07K 16/3069; C12N 5/0646; A61K 2239/39; C07K 2317/622; C07K 2319/03; C12N 2510/00

(57) ABSTRACT

Nucleic acid molecules encoding an IL-15 domain and a chimeric antigen receptor (CAR) that targets cells expressing prostate stem cell antigen (PSCA) are provided as well as polypeptides encoded thereby. Vectors and immune cells (e.g., NKT cells) containing the nucleic acid molecules also are disclosed, as well as methods for their use.

### CLAIM OF PRIORITY

This application claims the benefit of U.S. Provisional Application Ser. No. 63/610,651, filed on Dec. 15, 2023. The entire contents of the foregoing are incorporated herein by reference.

### SEQUENCE LISTING

This application contains a Sequence Listing which has been submitted electronically in XML format and is hereby incorporated by reference in its entirety. Said XML copy, created on Mar. 5, 2025, is named 40056-0084001_SL.xml and is 69,409 bytes in size.

### TECHNICAL FIELD

This disclosure concerns PSCA-specific chimeric antigen receptor (CAR)-engineered NK T cells, methods of formulating, and methods of use.

### BACKGROUND

Prostate stem cell antigen (PSCA) is highly expressed in various solid tumor cells, including pancreatic cancer, prostate cancer, and urinary bladder cancer but has limited expression in normal cells (Argani et al., (2001) Cancer Res. 61, 4320-4324; Abate-Daga et al., (2014) Hum Gene Ther. 25(12), 1003-1012). Pancreatic cancer (PC) remains the 4th leading cause of cancer-related deaths in the United States despite being the 10th most frequently diagnosed malignancy (Siegel et al., (2012) CA Cancer J. Clin. 62, 10-29). Most patients present with locally advanced or metastatic disease at diagnosis and are therefore not eligible for surgical resection. In addition, pancreatic cancer cells tend to be intrinsically resistant to chemo- and radiotherapy. Pancreatic cancer is one of the most aggressive solid tumors, with a high morbidity and mortality rate. PC accounts for 7% of all cancer deaths, and the general 5-year survival rate for PC patients is 10% (Islami F, et al. (2022) American Cancer Society's report on the status of cancer disparities in the United States, 2021. 72(2): 112-43). It is predicted that pancreatic ductal adenocarcinoma (PDAC) will be the second leading cause of cancer-related deaths by 2030 (Rahib L, et al. (2014) Projecting cancer incidence and deaths to 2030: the unexpected burden of thyroid, liver, and pancreas cancers in the United States. Cancer Res. 74(11): 2913-21). The poor prognosis of PC is associated with several factors, including the absence of specific symptoms leading to diagnosis at an advanced stage with local and/or distant metastases; high resistance of PC cells to existing standard chemotherapy; and a highly immunosuppressive and metabolically challenging tumor microenvironment (TME). To date, salvage chemotherapy regimens remain the best treatment for patients with advanced PC. Gemcitabine (2′,2′-difluorodeoxycytidine) is a nucleoside analog that represents a first-line intervention to treat advanced PDAC, but overall survival rates remain poor and there are few available options for patients who have failed gemcitabine-based therapy (Lee H S and Park S W (2016) Systemic Chemotherapy in Advanced Pancreatic Cancer. Gut Liver 10(3): 340-47; Binenbaum Y, Na'ara S, and Gil Z (2015) Gemcitabine resistance in pancreatic ductal adenocarcinoma. Drug Resist Updat 23:55-68). Importantly, the current standard of care is does not induce a proven survival benefit. Median survival is currently estimated to be 6-8 months (Cartwright et al., (2008) Cancer Control 15, 308-313). In recent years, substantial progress has been made with chimeric antigen receptor T-cell (CAR-T) therapies to treat hematologic malignancies, such as inducing remissions and improving long-term relapse-free survival in B-cell leukemia, lymphoma, and multiple myeloma patients. Unfortunately, the results of clinical trials suggest that chimeric antigen receptor T-cell therapy has limited success in solid tumors. Therefore, more effective immunotherapies are needed to treat these PSCA-positive cancers.

### SUMMARY OF THE DISCLOSURE

This application is based, at least in part, on the discovery that Natural Killer T cells (NKT cells) expressing a PSCA-targeted CAR and a soluble IL-15 mediate pancreatic cancer regression.

Type-I NKT cells are an evolutionarily conserved sub-lineage of T cells that express the invariant TCR alpha chain (Valpha24-Jalpha18), have characteristics that are intermediate between NK and T cells, and recognize self- and microbial-derived glycolipids presented by the monomorphic HLA class I molecule CD1d (Heczey A, Liu D, Tian G, et al. Invariant NKT cells with chimeric antigen receptor provide a novel platform for safe and effective cancer immunotherapy. Blood 2014; 124(18):2824-33). Unlike human leukocyte antigen molecules, which have genetic polymorphism and ubiquitous expression, the CD1d gene is monomorphic and presented only on a few cell types, thus limiting the toxicity of autologous or allogeneic NKT cells regardless of HLA allele expression (Ulanova M, Tarkowski A, Porcelli S A, et al. Antigen-specific regulation of CD1 expression in humans. J Clin Immunol 2000; 20(3):203-11). The application of NKT cells in CAR-based immunotherapy has distinct mechanistic advantages over bulky T cells. NKT cell infiltration of primary tumors correlates with better outcomes in different tumors (Tachibana T, Onodera H, Tsuruyama T, et al. Increased intratumor Valpha24-positive natural killer T cells: a prognostic factor for primary colorectal carcinomas. Clin Cancer Res 2005; 11(20):7322-7). Donor-derived NKT cells have been shown in several studies to inhibit graft-versus-host disease and preserve antitumor activity (Pillai A B, George T I, Dutt S, et al. Host NKT cells can prevent graft-versus-host disease and permit graft antitumor activity after bone marrow transplantation. Journal of immunology (Baltimore, Md: 1950) 2007; 178(10):6242-51). NKT cells traffic to solid tumors in response to chemokines produced by tumor cells, stromal cells, and tumor-associated macrophages (Metelitsa L S, Wu H W, Wang H, et al. Natural killer T cells infiltrate neuroblastomas expressing the chemokine CCL2. The Journal of experimental medicine 2004; 199(9):1213-21).

Described herein are human NKT cells expressing a CAR targeted to PSCA and at least a functional portion of human IL-15 (e.g., soluble human interleukin 15 (sIL15)). The soluble IL-15, e.g., co-expressed sIL-15, can enhance the anti-tumor and proliferative functions of NKT cells for sustained tumor control. The studies described herein demonstrate that PSCA CAR NKT cells expressing soluble human IL-15 (PSCA CAR_sIL15 NKT cells) exhibit durable antitumor efficacy in vitro and in vivo without causing significant toxicity in multiple models. Patients with relapsed PC that is resistant to first-line standard gemcitabine chemotherapy have few other therapeutic options. In this case, we validated the PSCA expression of gemcitabine-resistant cell lines and demonstrated that PSCA CAR_sIL15 NKT cells could control tumor progression well even though the gemcitabine-resistant cell lines exhibited more aggressive characteristics. Patients with recurrent PC have a very short survival time, especially with distant metastases, and NKT cells from pancreatic cancer patients treated with multiple lines of chemotherapy may not be able to prepare a qualified CAR-NKT cell product, so ready-to-use PSCA CAR_sIL15 NKT cell products may be able to maximize the survival benefit for patients. Off-the-shelf PSCA CAR_sIL15 NKT cells were validated to have superior anti-tumor function even after undergoing a round of freezing and thawing, with a lower risk of GvHD compared to PSCA CAR_sIL15 T cells from the same donor. These preclinical evaluations provide a solid basis for investigating PSCA CAR_sIL15 NKT cells for clinical application.

Provided herein are nucleic acid molecules comprising, or alternatively consisting essentially of, or alternatively consisting of a first nucleotide molecule encoding PSCA CAR and a second nucleotide molecule encoding an IL-15 domain (e.g., sIL-15). Also provided herein are nucleic acid molecules comprising, or alternatively consisting essentially of, or alternatively consisting of, a nucleotide molecule encoding a PSCA CAR and encoding an IL-15 (e.g., sIL-15) domain. The CAR comprises, or alternatively consists essentially of, or alternatively consists of a single chain variable fragment (scFv) targeting prostate stem cell antigen (PSCA), a spacer, a transmembrane domain, a co-stimulatory domain and a CD3% signaling domain. The nucleic acid molecule can be DNA or RNA.

Described herein are methods for making and using PSCA CAR NKT cells or other immune cells expressing a PSCA CAR and co-expressing an IL-15 domain (e.g., at least a portion of human IL-15, at least a portion of human IL-15Ra, or a fusion protein that includes at least a portion of human IL-15 and at least a portion of human IL-15Ra) to treat, for example, pancreatic cancer. The PSCA CAR NKT cells described herein possess potent antigen-specific anti-tumor efficacy in vitro and in vivo. The PSCA CAR NKT cells described herein also possess the potent antigen-specific anti-tumor efficacy.

Described herein are nucleic acid molecules comprising a nucleotide sequence encoding a CAR comprising an scFv targeting PSCA, a spacer, a transmembrane domain, a co-stimulatory domain, and a CD3ζ signaling domain, and a nucleotide sequence encoding a polypeptide comprising an IL-15 domain.

PSCA scFv

In some embodiments, the PSCA CAR comprises a PSCA scFv comprising or consisting of the amino acid sequence:

(SEQ ID NO: 1)
DIQLTQSPSTLSASVGDRVTITCSASSSVRFIHWYQQKPGKAPKRLI
YDTSKLASGVPSRFSGSGSGTDFTLTISSLQPEDFATYYCQQWGSSP
FTFGQGTKVEIKGSTSGGGSGGGSGGGGSSEVQLVEYGGGLVQPGGS
LRLSCAASGFNIKDYYIHWVRQAPGKGLEWVAWIDPENGDTEFVPKF
QGRATMSADTSKNTAYLQMNSLRAEDTAVYYCKTGGFWGQGTLVTVS
S,

VL followed by VH joined by a linker, with up to 5 (e.g., 1, 2, 3, 4, or 5 amino acid substitutions) or up to 10 single amino acid substitutions. In some embodiments, the amino acid substitutions are not in the CDRs.

In some embodiments, the CAR comprises a PSCA scFv comprising or consisting of the amino acid sequence:

(SEQ ID NO: 40)
EVQLVEYGGGLVQPGGSLRLSCAASGFNIKDYYIHWVRQAPGKGLEW
VAWIDPENGDTEFVPKFQGRATMSADTSKNTAYLQMNSLRAEDTAVY
YCKTGGFWGQGTLVTVSSGGGSGGGSGGGGSSDIQLTQSPSTLSASV
GDRVTITCSASSSVRFIHWYQQKPGKAPKRLIYDTSKLASGVPSRFS
GSGSGTDFTLTISSLQPEDFATYYCQQWGSSPFTFGQGTKVEIKGST
S,

VH followed by VL joined by a linker, with up to 5 (e.g., 1, 2, 3, 4, or 5 amino acid substitutions) or up to 10 single amino acid substitutions. In some embodiments, the amino acid substitutions are not in the CDRs.

In some embodiments, the PSCA scFv comprises a light chain variable region (VL) that is at least 95% identical to or includes up to 5 (e.g., 1, 2, 3, 4 or 5) single amino acid substitutions (preferably outside the CDRs, Kabat definition, underlined) compared to:

(SEQ ID NO: 32)
DIQLTQSPSTLSASVGDRVTITCSASSSVRFIHWYQQKPGKAPKRLI
YDTSKLASGVPSRFSGSGSGTDFTLTISSLQPEDFATYYCQQWGSSP
FTFGQGTKVEIKGST.

In some embodiments, the PSCA scFv comprises a heavy chain variable region (VH) that is at least 95% identical to or includes up to 5 (e.g., 1, 2, 3, 4 or 5) single amino acid substitutions (preferably outside the CDRs, Kabat definition, underlined) compared to:

(SEQ ID NO: 33)
EVQLVEYGGGLVQPGGSLRLSCAASGFNIKDYYIHWVRQAPGKGLEW
VAWIDPENGDTEFVPKFQGRATMSADTSKNTAYLQMNSLRAEDTAVY
YCKTGGFWGQGTLVTVSS.

The PSCA targeted CAR (also called “PSCA CAR”) or PSCA targeted polypeptide (also called “PSCA polypeptide”) described herein include a PSCA-targeting scFv, e.g., a PSCA scFv described above. In some embodiments, an scFv comprises the amino acid sequence:

(SEQ ID NO: 32)
DIQLTQSPSTLSASVGDRVTITCSASSSVRFIHWYQQKPGKAPKRLI
YDTSKLASGVPSRFSGSGSGTDFTLTISSLQPEDFATYYCQQWGSSP
FTFGQGTKVEIKGST

and the sequence

(SEQ ID NO: 33)
EVQLVEYGGGLVQPGGSLRLSCAASGFNIKDYYIHWVRQAPGKGLEW
VAWIDPENGDTEFVPKFQGRATMSADTSKNTAYLQMNSLRAEDTAVY
YCKTGGFWGQGTLVTVSS

(in either order) joined by a flexible linker.

In some embodiments, a useful flexible linker is 1, 2, 3, 4, 5, 6, 7, 8, 9, or 10 repeats of the sequence GGGS (SEQ ID NO:38). In some embodiments, a useful flexible linker is 1, 2, 3, 4, 5, 6, 7, 8, 9, or 10 repeats of the sequence GGGGS (SEQ ID NO:39). For example, a useful linker can comprise (G4S) 3 GGGGSGGGGSGGGGS (SEQ ID NO:37). Other useful linker include: SSGGGSGGGSGGGGSS (SEQ ID NO: 48) and SGGGSGGGSGGGGSS (SEQ ID NO: 49).

In some embodiments, the PSCA CAR comprises an scFv comprising or alternatively consisting essentially of, or yet further consisting of: a heavy chain (CDR) 1 (CDRH1) comprising DYYIH (SEQ ID NO: 50), an HC CDR 2 (CDRH2) comprising WIDPENGDTEFVPKFQG (SEQ ID NO: 51), and an HC CDR 3 (CDRH3) comprising GGF; a light chain (LC) complementarity-determining region (CDR) 1 (CDRL1) comprising SASSSVRFIH (SEQ ID NO: 53), an LC CDR 2 (CDRL2) comprising DTSKLAS (SEQ ID NO: 54), and an LC CDR 3 (CDRL3) comprising QQWGSSPFT (SEQ ID NO: 55).

In another aspect, the PSCA scFv comprises or consists of the amino acid sequence of SEQ ID NO: 1 an equivalent of each thereof, or a variant thereof having 1, 2, 3, 4, 5, or 6 amino acid substitutions, wherein the substitutions are conservative and not in the CDRs.

In another aspect, the PSCA scFv comprises or consists of the amino acid sequence of SEQ ID NO: 40, or an equivalent of each thereof or a variant thereof having 1, 2, 3, 4, 5, or 6 amino acid substitutions, wherein the substitutions are conservative and not in the CDRs.

### PSCA CAR

A useful PSCA CAR or PSCA polypeptide can comprise the amino acid sequence of SEQ ID NO:41 or SEQ ID NO:42 (mature CAR lacking a signal sequence). Any disclosed CAR or polypeptide can be expressed in a form that includes a signal sequence, e.g., a human GM-CSF receptor alpha signal sequence (MLLLVTSLLLCELPHPAFLLIP; SEQ ID NO: 36); a IgGk signal peptide (METDTLLLWVLLLWVPGSTG; SEQ ID NO:29); a IgG2 signal peptide (MGWSSIILFLVATATGVH; SEQ ID NO:30); a IL-2 signal peptide (MYRMQLLSCIALSLALVTNS; SEQ ID NO:31); MDWIWRILFLVGAATGAHS (SEQ ID NO: 35).

The CAR or polypeptide can be expressed with additional sequences that are useful for monitoring expression, for example, a T2A or P2A skip sequence and a truncated EGFR or truncated CD19 or LNGFR.

In some embodiments, the PSCA CAR comprises or consists of the amino acid sequence:

(SEQ ID NO: 41; includes signal sequence)
LLLVTSLLLCELPHPAFLLIPDIQLTQSPSTLSASVGDRVTITCSAS
SSVRFIHWYQQKPGKAPKRLIYDTSKLASGVPSRFSGSGSGTDFTLT
ISSLQPEDFATYYCQQWGSSPFTFGQGTKVEIKGSTSGGGSGGGSGG
GGSSEVQLVEYGGGLVQPGGSLRLSCAASGFNIKDYYIHWVRQAPGK
GLEWVAWIDPENGDTEFVPKFQGRATMSADTSKNTAYLQMNSLRAED
TAVYYCKTGGFWGQGTLVTVSSLEPKSCDKTHTCPPCPDPKGTFWVL
VVVGGVLACYSLLVTVAFIIFWVRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMK
GERRRGKGHDGLYQGLSTATKDTYDALHMQALPPR;
or
(SEQ ID NO: 42; no signal sequence)
DIQLTQSPSTLSASVGDRVTITCSASSSVRFIHWYQQKPGKAPKRLI
YDTSKLASGVPSRFSGSGSGTDFTLTISSLQPEDFATYYCQQWGSSP
FTFGQGTKVEIKGSTSGGGSGGGSGGGGSSEVQLVEYGGGLVQPGGS
LRLSCAASGFNIKDYYIHWVRQAPGKGLEWVAWIDPENGDTEFVPKF
QGRATMSADTSKNTAYLQMNSLRAEDTAVYYCKTGGFWGQGTLVTVS
SLEPKSCDKTHTCPPCPDPKGTFWVLVVVGGVLACYSLLVTVAFIIF
WVRSKRSRLLHSDYMNMTPRRPGPTRKHYQPYAPPRDFAAYRSRVKF
SRSADAPAYQQGQNQLYNELNLGRREEYDVLDKRRGRDPEMGGKPRR
KNPQEGLYNELQKDKMAEAYSEIGMKGERRRGKGHDGLYQGLSTATK
DTYDALHMQALPPR.

In some embodiments, the PSCA CAR comprises or consists of the amino acid sequence:

(SEQ ID NO: 43; includes signal sequence)
LLLVTSLLLCELPHPAFLLIPDIQLTQSPSTLSASVGDRVTITCSAS
SSVRFIHWYQQKPGKAPKRLIYDTSKLASGVPSRFSGSGSGTDFTLT
ISSLQPEDFATYYCQQWGSSPFTFGQGTKVEIKGSTSGGGSGGGSGG
GGSSEVQLVEYGGGLVQPGGSLRLSCAASGFNIKDYYIHWVRQAPGK
GLEWVAWIDPENGDTEFVPKFQGRATMSADTSKNTAYLQMNSLRAED
TAVYYCKTGGFWGQGTLVTVSSLEPKSCDKTHTCPPCPFWVLVVVGG
VLACYSLLVTVAFIIFWVRSKRSRLLHSDYMNMTPRRPGPTRKHYQP
YAPPRDFAAYRSRVKFSRSADAPAYQQGQNQLYNELNLGRREEYDVL
DKRRGRDPEMGGKPRRKNPQEGLYNELQKDKMAEAYSEIGMKGERRR
GKGHDGLYQGLSTATKDTYDALHMQALPPR;
or
(SEQ ID NO: 44; no signal sequence)
DIQLTQSPSTLSASVGDRVTITCSASSSVRFIHWYQQKPGKAPKRLI
YDTSKLASGVPSRFSGSGSGTDFTLTISSLQPEDFATYYCQQWGSSP
FTFGQGTKVEIKGSTSGGGSGGGSGGGGSSEVQLVEYGGGLVQPGGS
LRLSCAASGFNIKDYYIHWVRQAPGKGLEWVAWIDPENGDTEFVPKF
QGRATMSADTSKNTAYLQMNSLRAEDTAVYYCKTGGFWGQGTLVTVS
SLEPKSCDKTHTCPPCPFWVLVVVGGVLACYSLLVTVAFIIFWVRSK
RSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQE
GLYNELQKDKMAEAYSEIGMKGERRRGKGHDGLYQGLSTATKDTYDA
LHMQALPPR.

The PSCA CAR or polypeptide can comprise the amino acid sequence of any of SEQ ID NOs: 41-45 and 69, or can comprise an amino acid sequence that is at least 95%, 96%, 97%, 98% or 99% identical to SEQ ID NOs: 41-45 and 69. The CAR or polypeptide can comprise the amino acid sequence of any of SEQ ID NOs: 41-45 and 69, with up to 1, 2, 3, 4 or 5 amino acid changes (preferably conservative amino acid changes and/or without changes to the CDR regions). The CAR scFv can comprise SEQ ID NO:32 (VL) with up to 1, 2, 3, 4 or 5 amino acid changes (preferably conservative amino acid changes and/or without changes to the CDR regions) and SEQ ID NO: 33 (VH) with up to 1, 2, 3, 4 or 5 amino acid changes (preferably conservative amino acid changes and/or without changes to the CDR regions) joined by a flexible linker.

In some embodiments, the nucleic acid encoding the amino acid sequences described herein arel codon optimized.

The CAR or polypeptide described herein can include a spacer located between the PSCA targeting domain (i.e., a PSCA targeted ScFv or variant thereof) and the transmembrane domain. A variety of different spacers can be used. Some of them include at least portion of a human Fc region, for example a hinge portion of a human Fc region or a CH3 domain or variants thereof. Table 1 provides various spacers that can be used in the CARs described herein. The immunoglobulin derived sequences can include one or more amino acid modifications, for example, 1, 2, 3, 4 or 5 substitutions, e.g., substitutions that reduce off-target binding.

The spacer region can also comprise an IgG4 hinge region having the sequence ESKYGPPCPSCP (SEQ ID NO: 4) or ESKYGPPCPPCP (SEQ ID NO: 3). The spacer region can also comprise the hinge sequence ESKYGPPCPPCP (SEQ ID NO: 3) followed by the linker sequence GGGSSGGGSG (SEQ ID NO: 2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 12). Thus, the entire spacer region can comprise the sequence:

(SEQ ID NO: 79)
ESKYGPPCPPCPGGGSSGGGSGGQPREPQVYTLPPSQEEMTKNQVSL
TCLVKGFYPSDIAVEWESNGQPENNYKTTPPVLDSDGSFFLYSRLTV
DKSRWQEGNVFSCSVMHEALHNHYTQKSLSLSLGK.

A variety of transmembrane domains can be used in the CAR. In some cases, the transmembrane domain is a CD28 transmembrane domain that includes a sequence that is at least 90%, at least 95%, at least 98% identical to or identical to: FWVLVVVGGVLACYSLLVTVAFIIFWV (SEQ ID NO: 14). In some cases, the CD28 transmembrane domain has 1, 2, 3, 4 of 5 amino acid changes (preferably conservative) compared to SEQ ID NO:14. Table 2 includes examples of suitable transmembrane domains. Where a spacer region is present, the transmembrane domain(TM) is located carboxy terminal to the spacer region.

The costimulatory domain can be any domain that is suitable for use with a CD35 signaling domain. In some cases, the co-signaling domain is a CD28 co-signaling domain that includes a sequence that is at least 90%, at least 95%, at least 98% identical to or identical to: RSKRSRLLHSDYMNMTPRRPGPTRKHYQPYAPPRDFAAYRS (SEQ ID NO: 22). In some cases, the 4-1BB co-signaling domain has 1, 2, 3, 4 of 5 amino acid changes (preferably conservative) compared to SEQ ID NO: 22.

The costimulatory domain(s) are located between the transmembrane domain and the CD3ζ signaling domain. Table 3 includes examples of suitable costimulatory domains together with the sequence of the CD3ζ signaling domain.

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10 or 3-15 amino acids e.g., 3 amino acids, preferably glycine, (e.g., GGG) is can be positioned between the costimulatory domain and the CD3ζ signaling domain.

The CD3ζ signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 21). In some cases, the CD3ζ signaling has 1, 2, 3, 4 of 5 amino acid changes (preferably conservative) compared to SEQ ID NO: 21.

### Spacer

A nucleic acid encoding the CAR described herein can include a nucleic acid molecule encoding a spacer located between the PSCA targeting domain (i.e., a PSCA targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG |
| | | (SEQ ID NO: 2) |
| IgG4 hinge (S→P) | 12 aa | ESKYGPPCPPCP |
| (S228P) | | (SEQ ID NO: 3) |
| IgG4 hinge | 12 aa | ESKYGPPCPSCP |
| | | (SEQ ID NO: 4) |
| IgG4 hinge | 22 aa | ESKYGPPCPPCPGGGSSGG |
| (S228P) + linker | | GSG (SEQ ID NO: 5) |
| Also called HL | | |
| CD28 hinge | 39 aa | IEVMYPPPYLDNEKSNGTI |
| | | IHVKGKHLCPSPLFPGPSK |
| | | P (SEQ ID NO: 6) |
| CD8 hinge-48 aa | 48 aa | AKPTTTPAPRPPTPAPTIA |
| | | SQPLSLRPEACRPAAGGAV |
| | | HTRGLDFACD |
| | | (SEQ ID NO: 7) |
| CD8 hinge-45 aa | 45 aa | TTTPAPRPPTPAPTIASQP |
| | | LSLRPEACRPAAGGAVHTR |
| | | GLDFACD |
| | | (SEQ ID NO: 8) |
| IgG4(HL-CH3) | 129 aa | ESKYGPPCPPCPGGGSSGG |
| Also called | | GSGGQPREPQVYTLPPSQE |
| IgG4(HL-ΔCH2) | | EMTKNQVSLTCLVKGFYPS |
| (includes S228P | | DIAVEWESNGQPENNYKTT |
| in hinge) | | PPVLDSDGSFFLYSRLTVD |
| | | KSRWQEGNVFSCSVMHEAL |
| | | HNHYTQKSLSLSLGK |
| | | (SEQ ID NO: 9) |
| IgG4(L235E, | 229 aa | ESKYGPPCPSCPAPEFEGG |
| N297Q) | | PSVFLFPPKPKDTLMISRT |
| | | PEVTCVVVDVSQEDPEVQF |
| | | NWYVDGVEVHNAKTKPREE |
| | | QFQSTYRVVSVLTVLHQDW |
| | | LNGKEYKCKVSNKGLPSSI |
| | | EKTISKAKGQPREPQVYTL |
| | | PPSQEEMTKNQVSLTCLVK |
| | | GFYPSDIAVEWESNGQPEN |
| | | NYKTTPPVLDSDGSFFLYS |
| | | RLTVDKSRWQEGNVFSCSV |
| | | MHEALHNHYTQKSLSLSLG |
| | | K (SEQ ID NO: 10) |
| IgG4(S228P, | 229 aa | ESKYGPPCPPCPAPEFEGG |
| L235E,N297Q) | | PSVFLFPPKPKDTLMISRT |
| | | PEVTCVVVDVSQEDPEVQF |
| | | NWYVDGVEVHNAKTKPREE |
| | | QFQSTYRVVSVLTVLHQDW |
| | | LNGKEYKCKVSNKGLPSSI |
| | | EKTISKAKGQPREPQVYTL |
| | | PPSQEEMTKNQVSLTCLVK |
| | | GFYPSDIAVEWESNGQPEN |
| | | NYKTTPPVLDSDGSFFLYS |
| | | RLTVDKSRWQEGNVFSCSV |
| | | MHEALHNHYTQKSLSLSLG |
| | | K (SEQ ID NO: 11) |
| IgG4(CH3) | 107 aa | GQPREPQVYTLPPSQEEMT |
| Also called | | KNQVSLTCLVKGFYPSDIA |
| IgG4(ΔCH2) | | VEWESNGQPENNYKTTPPV |
| | | LDSDGSFFLYSRLTVDKSR |
| | | WQEGNVFSCSVMHEALHNH |
| | | YTQKSLSLSLGK |
| | | (SEQ ID NO: 12) |
| IgG1 Hinge | 16 aa | LEPKSCDKTHTCPPCP |
| | | (SEQ ID NO: 28) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The spacer region can also comprise an IgG4 hinge region having the sequence ESKYGPPCPSCP (SEQ ID NO: 4) or ESKYGPPCPPCP (SEQ ID NO: 3). The spacer region can also comprise the hinge sequence ESKYGPPCPPCP (SEQ ID NO: 3) followed by the linker sequence GGGSSGGGSG (SEQ ID NO: 2) followed by IgG4 CH3 sequence GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 12). Thus, the entire spacer region can comprise the sequence:

(SEQ ID NO: 9)
ESKYGPPCPPCPGGGSSGGGSGGQPREPQVYTLPPSQEEMTKNQ
VSLTCLVKGFYPSDIAVEWESNGQPENNYKTTPPVLDSDGSFFL
YSRLTVDKSRWQEGNVFSCSVMHEALHNHYTQKSLSLSLGK.

### Transmembrane Domain

A variety of nucleic acid molecules encoding the transmembrane domain of the CARs can be used in the CAR. In some aspects, a second spacer is located carboxy terminal to the transmembrane domain.

In some cases, the transmembrane domain nucleic acid molecule encodes a domain selected from a CD4 transmembrane domain, a CD8 transmembrane domain, a CD28 transmembrane domain, or a NKG2D transmembrane domain. In one aspect, the transmembrane domain encodes an amino acid sequence of SEQ ID NOS: 13-20 or 65, or an equivalent of each thereof. In some cases, the transmembrane domain has 1, 2, 3, 4 of 5 amino acid changes (preferably conservative) compared to SEQ ID NOS: 13-20 or 65, respectively.

In one aspect, the nucleic acid molecule encodes a CD28 transmembrane domain that includes a sequence that is at least 90%, at least 95%, at least 98% identical to or identical to: FWVLVVVGGVLACYSLLVTVAFIIFWV (SEQ ID NO: 14). In some cases, the CD28 transmembrane domain has 1, 2, 3, 4 of 5 amino acid changes (preferably conservative) compared to SEQ ID NO: 14.

Table 2 includes additional examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVTVAF |
| | | | IIFWV (SEQ ID NO: 14) |
| CD28(M) | NM_006139 | 28 aa | MFWVLVVVGGVLACYSLLVTVA |
| | | | FIIFWV (SEQ ID NO: 15) |
| CD4 | M35160 | 22 aa | MALIVLGGVAGLLLFIGLGIFF |
| | | | (SEQ ID NO: 16) |
| CD8tm | NM_001768 | 21 aa | IYIWAPLAGTCGVLLLSLVIT |
| | | | (SEQ ID NO: 17) |
| CD8tm2 | NM_001768 | 23 aa | IYIWAPLAGTCGVLLLSLVITL |
| | | | Y (SEQ ID NO: 18) |
| CD8tm3 | NM_001768 | 24 aa | IYIWAPLAGTCGVLLLSLVITL |
| | | | YC (SEQ ID NO: 19) |
| 41BB | NM_001561 | 27 aa | IISFFLALTSTALLFLLFFLTL |
| | | | RFSVV (SEQ ID NO: 20) |
| NKG2D | NM_007360 | 21 aa | PFFFCCFIAVAMGIRFIIMVA |
| | | | (SEQ ID NO: 34) |

### Costimulatory Domain

In the CARs of this disclosure, one or more polynucleotides encoding one or more costimulatory domains can be used, non-limiting examples of such include CD28, 4-1BB, 2B4, OX40, DAP10, or DAP12 or a variant thereof having 1-5 (e.g., 1 or 2) amino acid modifications. In a further aspect, the costimulatory domain comprises one or more of CD28, 4-1BB, OX40, or 2B4 costimulatory domains or a variant thereof having 1-5 (e.g., 1 or 2) amino acid modifications.

In a further aspect, the polynucleotide encodes a domain that is suitable for use with a CD3ζ signaling domain. In some cases, the co-signaling domain is a CD28 co-signaling or costimulatory domain that includes a sequence that is at least 90%, at least 95%, at least 98% identical to or identical to: RSKRSRLLHSDYMNMTPRRPGPTRKHYQPYAPPRDFAAYRS (SEQ ID NO: 22). In some cases, the 4-1BB co-signaling or costimulatory domain has 1, 2, 3, 4 of 5 amino acid changes (preferably conservative) compared to SEQ ID NO: 20, 23 or 24.

The costimulatory domain(s) are located between the transmembrane domain and the CD3ζ signaling domain. Table 3 includes additional examples of suitable costimulatory domains together with the sequence of the CD3ζ signaling domain.

TABLE 3
CD32 Domain and Examples of Costimulatory Domains

| Name | Accession | Length | Sequence |
|---|---|---|---|
| CD3ζ | J04132.1 | 113 aa | RVKFSRSADAPAYQQ |
| | | | GQNQLYNELNLGRRE |
| | | | EYDVLDKRRGRDPEM |
| | | | GGKPRRKNPQEGLYN |
| | | | ELQKDKMAEAYSEIG |
| | | | MKGERRRGKGHDGLY |
| | | | QGLSTATKDTYDALH |
| | | | MQALPPR |
| | | | (SEQ ID NO: 21) |
| CD28 | NM_006139 | 42 aa | RSKRSRLLHSDYMNM |
| | | | TPRRPGPTRKHYQPY |
| | | | APPRDFAAYRS |
| | | | (SEQ ID NO: 22) |
| CD28gg* | NM_006139 | 42 aa | RSKRSRGGHSDYMNM |
| | | | TPRRPGPTRKHYQPY |
| | | | APPRDFAAYRS |
| | | | (SEQ ID NO: 23) |
| 41BB | NM_001561 | 42 aa | KRGRKKLLYIFKQPF |
| | | | MRPVQTTQEEDGCSC |
| | | | RFPEEEEGGCEL |
| | | | (SEQ ID NO: 24) |
| OX40 | NM_003327 | 42 aa | ALYLLRRDQRLPPDA |
| | | | HKPPGGGSFRTPIQE |
| | | | EQADAHSTLAKI |
| | | | (SEQ ID NO: 25) |
| 2B4 | NM_016382 | 120 aa | WRRKRKEKQSETSPK |
| | | | EFLTIYEDVKDLKTR |
| | | | RNHEQEQTFPGGGST |
| | | | IYSMIQSQSSAPTSQ |
| | | | EPAYTLYSLIQPSRK |
| | | | SGSRKRNHSPSFNST |
| | | | IYEVIGKSQPKAQNP |
| | | | ARLSRKELENFDVYS |
| | | | (SEQ ID NO: 26) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD35 signaling domain and a short linker consisting of 2-10 or 3-15 amino acids e.g., 3 amino acids (e.g., GGG) is can be positioned between the costimulatory domain and the CD3ζ signaling domain.

### CD3ζ Signaling Domain

In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 21). In some cases, the CD3ζ signaling has 1, 2, 3, 4 of 5 amino acid changes (preferably conservative) compared to SEQ ID NO: 21. Variants of the CD3ζ signaling domain are shown in Table 4.

TABLE 4
CD3ζ Variants

| Name | Accession | Length | Sequence |
|---|---|---|---|
| CD3ζ | | 113 aa | RVKFSRSADAPAYQQGQNQL |
| variant | | | YNELNLGRREEYDVLDKRRG |
| | | | RDPEMGGKPRRKNPQEGLFN |
| | | | ELQKDKMAEAFSEIGMKGER |
| | | | RRGKGHDGLFQGLSTATKDT |
| | | | FDALHMQALPPR (SEQ ID |
| | | | NO: 56) |
| CD3ζ | | 113 aa | RVKFSRSADAPAYQQGQNQL |
| variant | | | YNELNLGRREEYDVLDKRRG |
| | | | RDPEMGGKPRRKNPQEGLFN |
| | | | ELQKDKMAEAFSEIGMKGER |
| | | | RRGKGHDGLYQGLSTATKDT |
| | | | YDALHMQALPPR (SEQ ID |
| | | | NO: 57) |
| CD3ζ | | 113 aa | RVKFSRSADAPAYQQGQNQL |
| variant | | | YNELNLGRREEYDVLDKRRG |
| | | | RDPEMGGKPRRKNPQEGLYN |
| | | | ELQKDKMAEAYSEIGMKGER |
| | | | RRGKGHDGLFQGLSTATKDT |
| | | | FDALHMQALPPR (SEQ ID |
| | | | NO: 58) |
| CD3ζ | | 113 aa | RVKFSRSADAPAYQQGQNQL |
| variant | | | YNELNLGRREEYDVLDKRRG |
| | | | RDPEMGGKPRRKNPQEGLYN |
| | | | ELQKDKMAEAFSEIGMKGER |
| | | | RRGKGHDGLFQGLSTATKDT |
| | | | FDALHMQALPPR (SEQ ID |
| | | | NO: 59) |
| CD3ζ | | 113 aa | RVKFSRSADAPAYQQGQNQL |
| variant | | | YNELNLGRREEYDVLDKRRG |
| | | | RDPEMGGKPRRKNPQEGLFN |
| | | | ELQKDKMAEAYSEIGMKGER |
| | | | RRGKGHDGLFQGLSTATKDT |
| | | | FDALHMQALPPR (SEQ ID |
| | | | NO: 60) |
| CD3ζ | | 113 aa | RVKFSRSADAPAYQQGQNQL |
| variant | | | YNELNLGRREEYDVLDKRRG |
| | | | RDPEMGGKPRRKNPQEGLFN |
| | | | ELQKDKMAEAFSEIGMKGER |
| | | | RRGKGHDGLYQGLSTATKDT |
| | | | FDALHMQALPPR (SEQ ID |
| | | | NO: 61) |
| CD3ζ | | 113 aa | RVKFSRSADAPAYQQGQNQL |
| variant | | | YNELNLGRREEYDVLDKRRG |
| | | | RDPEMGGKPRRKNPQEGLFN |
| | | | ELQKDKMAEAFSEIGMKGER |
| | | | RRGKGHDGLFQGLSTATKDT |
| | | | YDALHMQALPPR (SEQ ID |
| | | | NO: 62) |

### Self-Cleaving Peptide

In some aspects, a self-cleaving peptide located between the CAR and a co-expressed polypeptide, e.g., sIL-15. Non-limiting examples of such include a 2A self-cleaving peptide. A 2A self-cleaving peptide refers to a class of 18-22 aa-long peptides, which can induce the cleaving of the recombinant protein in a cell. In some embodiments, the 2A self-cleaving peptide is selected from P2A, T2A, E2A, F2A and BmCPV2A. (Wang et al. 2A self-cleaving peptide-based multi-gene expression system in the silkworm Bombyx mori. Sci Rep. 5:16273, 2015). A non-limiting example includes a polypeptide that is at least 95%, 96%, 97%, 98%, or 99% identical or identical to LEGGGEGRGSLLTCGDVEENPGPR; SEQ ID NO: 27). Other ribosomal skip sequences useful in a CAR or peptide described herein include T2At having a sequence that is at least 95%, 96%, 97%, 98%, or 99% identical or identical to: EGRGSLLTCGDVEENPGP (SEQ ID NO: 46) or P2A having a sequence that is at least 95%, 96%, 97%, 98%, or 99% identical or identical to: GSGATNFSLLKQAGDVEENPGP (SEQ ID NO: 47). In some cases, the ribosomal skip sequence has 1, 2, 3, 4 of 5 amino acid changes (preferably conservative) compared to SEQ ID NO: 27 or 46 or 47.

### Signal Peptide

The nucleic acid molecule encoding the CAR can further comprise a polynucleotide encoding a signal peptide to direct cell surface expression, e.g., a human GM-CSF receptor alpha signal sequence (MLLLVTSLLLCELPHPAFLLIP; SEQ ID NO: 36); a IgGk signal peptide (METDTLLLWVLLLWVPGSTG; SEQ ID NO: 29); a IgG2 signal peptide (MGWSSIILFLVATATGVH; SEQ ID NO: 30); a IL-2 signal peptide (MYRMQLLSCIALSLALVTNS; SEQ ID NO: 31) or MDWIWRILFLVGAATGAHS (SEQ ID NO: 35) an equivalent of each thereof. When co-expressed polypeptide, sIL-15, is expressed with the CAR and is meant to be secreted or present on the cell surface, it can include a signal peptide.

### IL-15 Domain

In some embodiments, the IL-15 domain includes at least a functional portion of human IL-15 proprotein (e.g., amino acids 30-162 human IL-15 isoform I; GenBank NP_0056). The sequence of the full length proprotein is: GIHVFILGCFSAGLPKTEANWVNVISDLKKIEDLIQSMHIDATLYTESDVHPSCKVTA MKCFLLELQVISLESGDASIHDTVENLIILANNSLSSNGNVTESGCKECEELEEKNIKE FLQSFVHIVQMFINTS (human IL-15 proprotein; SEQ ID NO: 63). Thus, the IL-15 domain can include at least a functional portion of human IL-15 (e.g., amino acids 49-162 human IL-15 isoform I; GenBank NP_0056): NWVNVISDLKKIEDLIQSMHIDATLYTESDVHPSCKVTAMKCFLLELQVISLESGDAS IHDTVENLIILANNSLSSNGNVTESGCKECEELEEKNIKEFLQSFVHIVQMFINTS (human IL-15; SEQ ID NO: 64). In some embodiments, the IL-15 domain comprises or consists of: MRISKPHLRSISIQCYLCLLLNSHFLTEAGIHVFILGCFSAGLPK TEANWVNVISDLKKIEDLIQSMHIDATLYTESDVHPSCKVTAMKCFLLELQVISLESG DASIHDTVENLIILANNSLSSNGNVTESGCKECEELEEKNIKEFLQSFVHIVQMFINTS (human IL-15 preproprotein SEQ ID NO: 65). In some cases, the IL-15 domain has 1, 2, 3, 4 of 5 amino acid substitution (preferably conservative) compared to SEQ ID NO: 63, 64 or 65. In some embodiments, the IL-15 is a variant IL-15 comprising one or more mutations selected from L45D, L45E, S51D, L52D, N72D, N72E, N72A, N72S, N72Y and N72P, wherein the first letter indicates the original amino acid residue, the last letter indicates the mutated amino acid residue, and the number in the center indicates the amino acid residue number in or aligned to the sequence of In some embodiments, the nucleotide sequence encoding the IL-15 domain is codon optimized.

In some embodiments, the IL-15 domain includes of human IL-15 receptor alpha subunit isoform I (e.g., amino acids 31-205 of GenBank NP_002180) or can be any domain having that structure or function, including but not limited to, soluble IL-15 (sIL-15), membrane bound IL-15 (mbIL-15 or mIL-15), sIL-15 complex IL-15Rα (sIL-15c), and mbIL-15 complexed with IL-15Rα (mbIL-15c or mIL-15c), and mimetics thereof.

In some embodiments, the IL-15 is membrane bound and includes a transmembrane domain, e.g., a transmembrane domain that is at least 90%, at least 95%, at least 98% identical to or identical to: VAISTSTVLLCGLSAVSLLACYL (SEQ ID NO: 66). In some cases, the transmembrane domain within the IL-15 domain has 1, 2, 3, 4 of 5 amino acid changes (preferably conservative) compared to SEQ ID NO: 66.

### Preparation of Cell Populations

Also provided are vectors comprising, or alternatively consisting essentially of, or yet further consisting of a nucleic acid molecule as disclosed herein. In some embodiments, a vector or nucleic acid molecule further comprises a regulatory sequence operatively linked to one or more elements of the nucleic acid molecule that direct the expression or the replication of the nucleic acid molecule. In some embodiments the vector or nucleic acid molecule includes sequences that direct expression of the CAR.

Also described are expression vectors comprising a nucleic acid molecule described herein and a population of human NKT cells transduced by the vector or harboring a nucleic acid molecule (e.g., mRNA) descried herein.

The CARs or polypeptides described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform suitable human NKT cells.

The CARs or polypeptides can be transiently expressed in a NKT cells population by an mRNA encoding the CAR or polypeptide. The mRNA can be introduced into the NKT cells by electroporation (Wiesinger et al. 2019 Cancers (Basel) 11:1198).

Treatment with PSCA CAR NKT Cells

Also described is a method of treating a solid tumor or a cancer in a patient comprising administering a population of human NKT cells transduced by a vector comprising a nucleic acid molecule described herein or harboring a nucleic acid molecule described herein, wherein the solid tumor or cancer comprises cells expressing PSCA. In various embodiments: the population of human NKT cells is administered locally or systemically and is administered by single or repeat dosing.

Also provided are methods of inhibiting the growth of a cancer or solid tumor expressing PSCA or a tissue comprising a cancer cell expressing PSCA, the method comprising, or alternatively consisting essentially of, or alternatively consisting of, contacting the cancer or solid tumor expressing PSCA or a tissue with a cell or a population of cells disclosed herein.

Also provided are methods of treating a cancer that expresses PSCA in a subject in need thereof, comprising, or alternatively consisting essentially of, or alternatively consisting of, administering to the subject a cell or a population of cells disclosed herein, thereby treating the cancer.

Also provided are methods for treating a solid tumor or cancer in a patient in need thereof, comprising, or alternatively consisting essentially of, or alternatively consisting of, administering a population of autologous or allogenic NKT cells transduced by a vector comprising, or alternatively consisting essentially of, or alternatively consisting of, a nucleic acid molecule disclosed herein, and wherein the solid tumor or cancer comprises cells expressing PSCA.

Also described herein are methods of treating a solid tumor or a method of reducing or eliminating PSCA-positive cells. Also described herein is a method of treating PSCA-positive cancers or disorders (including, e.g., pancreatic cancer, prostate cancer, and urinary bladder cancer) in a patient comprising administering a population of autologous or allogeneic human NKT cells transduced by a vector comprising a nucleic acid molecule described herein, wherein the PSCA-positive cancers or disorders comprise cells expressing PSCA. In various embodiments: human NKT cells expressing a chimeric antigen receptor or polypeptide described herein are administered locally or systemically; the PSCA-expressing target cells are cancerous cells; and the human NKT cells expressing chimeric antigen receptor or polypeptide are administered by single or repeat dosing.

Also described herein are methods for using PSCA CAR NKT cells as anti-cancer agents selective against PSCA-positive cell; also described herein are methods of decreasing the population of non-cancerous PSCA-positive cells. In some embodiments, described herein is a method of reducing or eliminating PSCA-positive cells in a subject comprising administering a population of autologous or allogeneic human NKT cells transduced by a vector comprising the nucleic acid molecule encoding a PSCA CAR.

Also provided are methods of reducing or eliminating PSCA-positive cells in a subject comprising administering a population of autologous or allogeneic human NKT cells transduced by a vector comprising a nucleic acid molecule disclosed herein.

In some embodiments, described herein is a method of increasing survival of a subject having cancer comprising administering a composition comprising a PSCA CAR NKT cells described herein.

In some embodiments, described herein is a method of reducing or ameliorating a symptom associated with a cancer in a patient comprising administering a composition comprising a PSCA CAR NKT cell described herein.

Administration of the cells or compositions can be performed in one dose, continuously or intermittently throughout the course of treatment and an effective amount to achieve the desired therapeutic benefit is provided. In a further aspect, the cells and composition of the disclosure can be administered in combination with other treatments.

The compositions as described herein can be administered as first line, second line, third line, fourth line, or other therapy and can be combined with another anti-cancer therapy. They can be administered sequentially or concurrently as determined by the treating physician.

In some embodiments, the composition or method as disclosed herein can be combined with therapies that may upregulate the expression of a tumor or other antigen to which the CAR binds. In some embodiments, some clinical drugs can increase targeted antigens.

In some embodiments, the compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety.

Other features and advantages of the invention will be apparent from the following detailed description and figures, and from the claims.

### DESCRIPTION OF DRAWINGS

FIGS. 1A-1F. NKT cells expressing PSCA CAR_sIL15 construct demonstrated superior in vitro expansion and expressed low levels of exhaustion markers. FIG. 1A, Schematic structures of the clinical grade vectors. FIG. 1B, Representative flow cytometric analysis of PSCA CAR_sIL15/sIL15 NKT cells demonstrating the proportion of CD3 and iNKT (TCR Vα24-Jα18) expression 2 days after transduction. FIG. 1C, The transduction ratio of PSCA CAR_sIL15/sIL15 NKT cells were detect by measuring tEGFR expression after 2 days of transduction, analyzed using flow cytometry. FIG. 1D, The basal apoptosis of PSCA CAR_sIL15/sIL15 NKT cells measured by annexin V and 7-AAD staining 2 days post-transduction. FIG. 1E, Quantification of total PSCA CAR_sIL15/sIL15 NKT cell fold expansion following 12 days of secondary expansion (mean±SD, n=3). The number of NKT cells was determined using a Muse Cell Analyzer (Luminex Corporation, Austin, TX). Analysis of live and dead cells was performed with the Muse Count & Viability Kit. Not significant (ns, Student t test). FIG. 1F, The exhaustion markers LAG-3, PD-1, and TIM-3 of PSCA CAR_sIL15/sIL15 NKT cells were detected by flow cytometry. The results are displayed as mean±SD (n=3). Not significant (ns, two-way ANOVA).

FIGS. 2A-2F. PSCA CAR_sIL15 NKT cells demonstrated PSCA⁺ PC cell-specific activation. FIG. 2A, Surface expression of PSCA on PC cell lines measured using flow cytometry. FIG. 2B, Expression of activation markers CD69 and CD25 on PSCA CAR_sIL15/sIL15 NKT cells following a 24 h co-incubation with Capan-1, MIA Paca-2, or BxPC-3 (gated on EGFR⁺ cells). The data represent mean±SD (n=3). FIG. 2C, flow cytometric analysis showing the expression of CD69 and CD25 on PSCA CAR_sIL15/sIL15 NKT cells after co-incubation with target cells. FIG. 2D, Flow cytometric analysis (left) showing CD107a expression on PSCA CAR_sIL15/sIL15 NKT cells after co-incubation with target cells and its quantitative analysis (right). FIG. 2E, Flow cytometric analysis (left) showing TNF-α expression in PSCA CAR_sIL15/sIL15 NKT cells after co-incubation with target cells and its quantitative analysis (right). FIG. 2F, flow cytometric analysis (left) showing IFN-γ expression in PSCA CAR_sIL15/sIL15 NKT cells after co-incubation with target cells and its quantitative analysis (right).

FIGS. 3A-3B. PSCA CAR_sIL15 NKT cells demonstrated potent and specific cytotoxicity against PC cell lines in vitro. FIG. 3A, RTCA results of cytotoxicity of PSCA CAR_sIL15/sIL15 NKT cells against PSCA⁺ Capan-1, MIA Paca-2, and Aspc-1 or PSCA⁻ Panc-1 and BxPC-3 tumor cells with an E:T ratio of 1:1. FIG. 3B, After 90 hours of co-incubation, representative images of the killing of PSCA CAR_sIL15/sIL15 NKT cells targeting different PC cells.

FIGS. 4A-4J. PSCA CAR_sIL15 NKT cells showed superior therapeutic activity in metastatic mouse models without causing significant toxicity. FIG. 4A, Schematic diagram of treatment with PSCA CAR_sIL15 NKT cells in a human metastatic PC model established by injection of Capan-1_luc cells into NSG mice. The figure was created in BioRender (biorender.com/). FIG. 4B, Tumor growth in NSG mice inoculated with Capan-1_luc was monitored by changes in tumor bioluminescence, with colors indicating the intensity of the luminescence. FIG. 4C, Summary of mouse tumor burden changes of each treatment group from FIG. 4B up to day 32. The results are displayed as mean±SD (n=4). *P<0.05, **P<0.01 (two-way ANOVA). FIG. 4D, Overall Kaplan-Meier survival curve. **P<0.01 (log-rank test). FIG. 4E, Schematic diagram of treatment with PSCA CAR_sIL15 NKT cells in a human metastatic PC model established by injection of MIA PaCa-2_luc cells into NSG mice. The picture was created with BioRender.com. FIG. 4F, Representative images of the pancreas and liver of each group in the MIA PaCa-2-based PC mouse model at the endpoint of the in vivo experiments. Red arrows mark metastatic tumors on the liver. FIG. 4G, Radiance fold changes of each treatment group measured at the indicated time points. The results are displayed as mean±SD. **P<0.01, ***P<0.001 (two-way ANOVA). FIG. 4H, The growth and staging of the tumor are monitored by bioluminescence imaging. FIG. 4I, Overall Kaplan-Meier survival curve. *P<0.05, ***P<0.001 (log-rank test). FIG. 4J, Assessment of blood cell populations on day 15 after inoculation (day 12 after treatment). Values represent mean±SD. Not significant (ns, two-way ANOVA)

FIGS. 5A-5F. PSCA CAR_sIL15 NKT cells eliminate PC cells in an orthotopic tumor model and maintain long-term tumor-free survival. FIG. 5A, Schematic diagram of treatment with PSCA CAR_sIL15 NKT cells in a human orthotopic PC model established by intrapancreatic injection of MIA PaCa-2_luc cells into NSG mice. The figure was created in BioRender (biorender.co). FIG. 5B, Representative images of the pancreas and liver of each group in the MIA PaCa-2-based PC mouse model at the endpoint of the in vivo experiments. Red arrows mark metastatic tumors on the liver. FIG. 5C, Summary of mouse tumor burden changes of each treatment group. The results are displayed as mean±SD. **P<0.01, ****P<0.0001 (two-way ANOVA). FIG. 5D, The growth and staging of the tumor are monitored by bioluminescence imaging. FIG. 5E, Overall Kaplan-Meier survival curve. ****P<0.0001 (log-rank test). FIG. 5F, Assessment of blood cell populations on day 15 after inoculation (day 12 after treatment). Values represent mean±SD. Not significant (ns, two-way ANOVA).

FIGS. 6A-6H. Gemcitabine resistance in PC can be overcome by PSCA CAR_sIL15 NKT cells. FIG. 6A, Surface expression of PSCA (blue solid histograms) on gemcitabine-resistant cell lines (Capan-1 GR and MIA Paca-2 GR) in comparison with original cell lines (red solid histograms) measured using flow cytometry. FIG. 6B, RTCA results demonstrated the effect of different concentrations of gemcitabine on gemcitabine-resistant cell lines (Capan-1 GR and MIA Paca-2 GR) and original cell lines. FIG. 6C, Cytotoxicity of PSCA CAR_sIL15/sIL15 NKT cells against gemcitabine-resistant cell lines (Capan-1 GR and MIA Paca-2 GR) and primary cell lines at an E:T ratio of 1:2 using RTCA assay. Expression of T cell activation markers CD69 (FIG. 6D) and CD25 (FIG. 6E) on PSCA CAR_sIL15/sIL15 NKT cells following a 24 h co-incubation with Capan-1 GR, Capan-1, MIA Paca-2 GR, and MIA Paca-2 (gated on EGFR⁺ cells). The data represent mean±SD (n=3). FIG. 6F, Capan-1 GR_luc and its control Capan-1_luc were injected by intraperitoneal injection (2×10⁵ cells/mouse). Three days after tumor inoculation, tumor engraftment was confirmed using bioluminescence imaging (BLI). Mice with established tumors were randomly assigned to control or treatment groups and then given by IP (3×10⁶ CAR⁺ CAR-sIL15 NKT/sIL15 NKT cells) and intravenous injection (IV; 1.5×10⁶ CAR⁺ CAR-sIL15 NKT/sIL15 NKT cells). Summary of mouse tumor burden changes of each treatment group. The results are displayed as mean±SD. *P<0.05, ***P<0.001 (two-way ANOVA). FIG. 6G, The growth and staging of the tumor are monitored by bioluminescence imaging. FIG. 6H, Overall Kaplan-Meier survival curve. **P<0.01 (log-rank test).

FIGS. 7A-7J. Off-the-shelf PSCA CAR_sIL15 NKT cells demonstrated excellent antitumor function without causing GvHD. FIG. 7A, RTCA results of cytotoxicity of off-the-shelf PSCA CAR_sIL15/sIL15 NKT cells against PSCA⁺ Capan-1 and MIA Paca-2 tumor cells with an E:T ratio of 1:1. FIG. 7B, The growth and staging of the tumor are monitored by bioluminescence imaging. FIG. 7C, Summary of mouse tumor burden changes of each treatment group. The results are displayed as mean±SD. ***P<0.001 (two-way ANOVA). FIG. 7D, Overall Kaplan-Meier survival curve. ***P<0.001 (log-rank test). FIG. 7E, Schematic diagram of treatment with PSCA CAR_sIL15 NKT/T cells in a Capan-1-based metastatic PC humanized mice model. The figure was created in BioRender (biorender.com). FIG. 7F, Percentage of human CD45, CD3, CD4, CD8, CD56, and CD19 positive cells in the peripheral blood of NSG SGM3 mice were measured by flow cytometry on day 14 after PBMC injection. FIG. 7G, The growth and staging of the tumor are monitored by bioluminescence imaging. H, Radiance fold changes of each treatment group measured on day 24. FIG. 7I, Clinical GvHD scores observed on day 42. ***P<0.001 (Student t test). FIG. 7J, Spleen dimensions of PSCA CAR_sIL15 T (top)/NKT (bottom) cell-treated mice measured at the end of the experiments

FIG. 8. After 90 hours of c-incubation, original images of PSCA CAR_sIL15 MKT cells versus sIL15 NKT cells killing target cells.

FIG. 9. Mouse body weight after treatment with PSCA CAR_sIL15 NKT cells in a human metastatic PC model established by injection of Capan-1_luc cells into NSG mice.

FIG. 10. Images of gemcitabine-resistant cell lines (Capan-1 GR and MIA Paca-2 GR) and original cell lines after co-culture with different concentrations of gemcitabine and control 0.01% DMSO for 72 hours.

FIG. 11. RTCA results show the effect of different concentrations of gemcitabine on gemcitabine-resistant cell lines (Capan-1 GR and MIA Paca-2 GR) and original cell lines treated for 1 hour and 3 hours, respectively.

FIG. 12. Expression of CD107a (a surrogate marker for degranulation), intracellular cytokines TNF-α and IFN-γ (gated to EGFR⁺ cells) was detected in PSCA CAR_sIL15 NKT cells and sIL15 NKT cells with Capan-1 GR, Capan-1 MIA Paca-2 GR and MIA Paca-2 after 24 hours of co-culture using flow cytometry. Data represent mean±SD (n=3).

FIG. 13. Amino acid sequence of the PSCA CAR used in the studies described in FIGS. 1-6. The various domains are indicated. SEQ ID NO: 42 is the sequence lacking the signal sequence. SEQ ID NO: 41 includes the signal sequence.

FIG. 14. Amino acid sequence a PSCA CAR lacking the DPKGY (SEQ ID NO: 68) present in the PSCA CAR of FIG. 13 between the IgG1 hinge and the CD28 TM. The various domains are indicated. SEQ ID NO: 69 is the sequence lacking the signal sequence. SEQ ID NO: 45 includes the signal sequence.

### DETAILED DESCRIPTION

### Examples

The invention is further described in the following examples, which do not limit the scope of the invention described in the claims.

### Materials and Methods

The following materials and methods were used in the Examples set forth herein.

### Cell Lines

PSCA+ cell lines, Capan-1, MIA PaCa-2, and AsPC-1 cells, and PSCA″ cell lines, PANC-1 and BxPC-3 were cultured in DMEM medium containing 10% fetal bovine serum (FBS; Thermo Fisher, Waltham, MA, USA). All cell lines were verified before use.

### Generation of PSCA CAR_sIL15 NKT Cells

Anti-PSCA single-chain fragment variable was grafted into a second-generation CAR with an IgG1 hinge, CD28 transmembrane region and co-stimulatory domain, and intracellular CD3ζ. Because IL15 has been reported to improve NKT cell persistence, tumor site localization, and tumor control in vivo (Xu X, et al. (2019) NKT Cells Coexpressing a GD2-Specific Chimeric Antigen Receptor and IL15 Show Enhanced In Vivo Persistence and Antitumor Activity against Neuroblastoma. Clinical cancer research 25(23):7126-38), a soluble IL15 (s15) was incorporated into the PSCA CAR construct, with the PSCA CAR gene linked to a EGFRt by a T2A sequence for further translational and clinical research.

As we previously reported, retrovirus was generated using lipofectamine 3000 (ThermoFisher, Carlsbad, CA) by transient transfection of GP2-293T cells (Takara, San Francisco, CA) with pRD114-TR plasmid (Teng K-Y, et al. (2022) Off-the-Shelf Prostate Stem Cell Antigen-Directed Chimeric Antigen Receptor Natural Killer Cell Therapy to Treat Pancreatic Cancer. Gastroenterology 162(41319-33). The viral supernatants were collected at 48 h after transfection, filtered through a 0.45 μm filter, aliquoted, and stored at −80° C.

Human donor peripheral blood leukocytes from healthy donors were obtained from the City of Hope Michael Amini Transfusion Medicine Center. Peripheral blood mononuclear cells (PBMCs) were obtained from collected blood leukocytes using Ficoll-Paque Plus (GE Healthcare, Boston, MA, USA). NKT cells were isolated from PBMCs using anti-iNKT microbeads (Miltenyi Biotec, Bergisch Gladbach, Germany) and the negative PBMC fraction was irradiated (25 Gy) and aliquoted. Irradiated autologous PBMC loaded with α-galactosylceramide (α-GalCer, 100 ng/ml; Kyowa Hakko Kirin) was used to stimulate NKT cells in a 1:1 ratio. NKT cells were cultured in RPMI 1640 medium supplemented with 5% human serum (Sigma-Aldrich, St. Louis, MO) and IL-2 (100 IU/mL; National Institutes of Health, Bethesda, MD). NKT cells were expanded for 10-12 days and then restimulated with α-GalCer-loaded (100 ng/ml) irradiated autologous PBMC in a 2:1 ratio. Then, NKT cells were transduced with retrovirus using Retronectin (Takara, San Francisco, CA) according to the manufacturer's instructions and further expanded in RPMI 1640 medium supplemented with 5% human serum in the presence of IL-2.

### Flow Cytometry-based Assay

The cell lines were stained with PE-conjugated mouse anti-human PSCA antibody (clone: 7F5; Santa Cruz Biotechnology) and isotype antibody (BioLegend) to determine the PSCA antigen expression level. TCR Vα24-Jα18 (INKT cell; clone: 6B11), CD3 (clone: OKT3), EGFR (clone: AY13), CD56 (clone: QA17A16), CD8 (clone: SK1), CD4 (clone: A161A1), CD19 (clone: SJ25C1), TIM-3 (clone: F38-2E2), PD-1 (clone: EH12.1), LAG-3 (clone: 7H2C65), and CD45 (clone: HI30) antibodies were all purchased from BioLegend or BD Biosciences. PSCA CAR_sIL15 NKT/sIL15 NKT cells (4×10⁵) were co-incubated with equal tumor cells for 24 h, then the activation markers CD69 (clone: FN50; BioLegend) and CD25 (clone: BC96; BioLegend) were detected. The data were acquired with LSRFortessa™ X-20 Cell Analyzer (BD Biosciences) and analyzed with FlowJo software version 10 (Tree Star).

### Apoptosis Assay

Annexin V/7-AAD (BioLegend) staining was used to examine apoptotic cell death, and PSCA CAR_sIL15 NKT/sIL15 NKT cells (1×10⁶) were harvested and stained with according to the manufacturer's instructions before being subjected to flow cytometry analysis to detect apoptosis.

### Degranulation and Intracellular Cytokine Staining Assays

Degranulation and intracellular cytokine staining assays were performed by co-incubating 4×10⁵ CAR-sIL15 NKT/sIL15 NKT cells with 2×10⁵ indicated target cells in the presence of 1:50 PE/Cyanine7-conjugated CD107a antibody (clone: H4A3; BioLegend), GolgiPlug (BD Biosciences) and GolgiStop (BD Biosciences) were added 1 hour after plating according to the manufacturer's instructions. After further culture for 4 h, cells were incubated with antibodies for surface markers and permeabilized for 20 minutes using Fixation/Permeablization Kit (BD Biosciences), followed by staining with TNF-α (clone: MAb11; BD Biosciences) and IFN-γ (clone: 4S; B3; BD Biosciences) antibodies.

### Cytolysis Assay (Real-Time Cell Analysis)

The cytolytic capability of PSCA CAR_sIL15 NKT/sIL15 NKT cells against PSCA+ tumor cells was assessed using a real-time cell analysis (RTCA) assay (ACEA Bioscience, xCELLigence RTCA MP, San Diego, CA) according to the manufacturer's instructions. Target cells (2×10³) were seeded on RTCA plates in triplicate and incubated in culture medium at 37° C., 5% CO₂ conditions. After 20-24 hours, PSCA CAR_sIL15 NKT/sIL15 NKT cells were added to the target cells at the indicated E:T ratios, and data were collected at 15-minute intervals. The xCELLigence MP system continuously monitored cell growth for 72 hours.

### Mouse Xenograft Models

For the PC metastasis model, male and female 8-12 weeks-old NOD SCID gamma (NSG) mice (NOD.Cg-Prkdc^{scid} Il2rg^{tm1Wj1}/SzJ; Jackson Laboratory, Bar Harbor, ME) were transplanted with Capan-1 or MIA PaCa-2 PC cell lines expressing the fluorescein_ZsGreen gene (Capan-1_luc or MIA PaCa-2_luc) by intraperitoneal (IP) injection (2×10⁵ cells/mouse). For the PC orthotopic model, 40 μL of 2×10⁵ MIA PaCa-2_luc tumor cells were surgically injected in the mouse pancreas. For the gemcitabine-resistant PC model, Capan-1 GR_luc and its control Capan-1_luc were injected by intraperitoneal injection (2×10⁵ cells/mouse). Prior to any treatment, tumor engraftment was confirmed using bioluminescence imaging (BLI). Mice with established tumors were randomly assigned to control or treatment groups and then given by IP (3×10⁶ CAR⁺ CAR-sIL15 NKT/sIL15 NKT cells) and intravenous injection (IV; 1.5×10⁶ CAR⁺ CAR-sIL15 NKT/sIL15 NKT cells). Tumor burden was evaluated using BLI, and body mass and survival were monitored. All animal experiments were performed in accordance with Animal Research Reporting In Vivo Experiments (ARRIVE), federal, state, and local guidelines, with the training and approval of City of Hope Animal Care and Use Committee.

### Graphs and Statistical Analysis

Graphs and data analyses were performed using GraphPad Prism Software version 8.3.0. Some of these graphs were obtained and modified from Servier Medical Art. Unless otherwise stated, all data are representative of at least three independent experiments. All data are presented as mean±SD. Significant differences were analyzed by Student t test, one-way analysis of variance, two-way analysis of variance, or log-rank test. P-values are represented as either not significant (ns), *P<0.05, **P<0.01, ***P<0.001, or ****P<0.0001.

### Example 1: NKT Cells Expressing PSCA CAR_sIL15 Construct Demonstrated Superior In Vitro Expansion and Expressed Low Levels of Exhaustion Markers

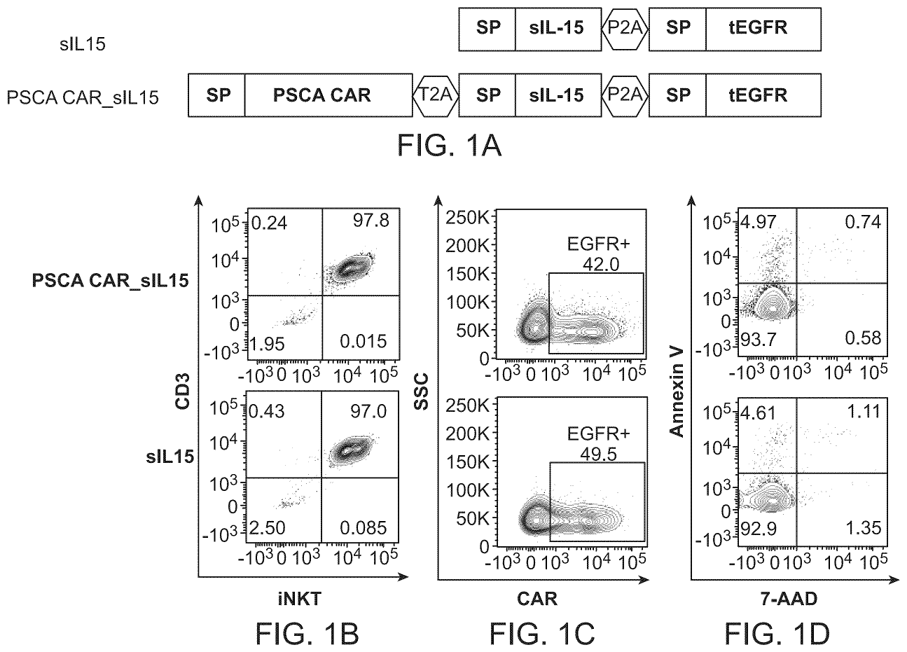

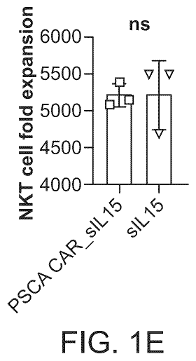

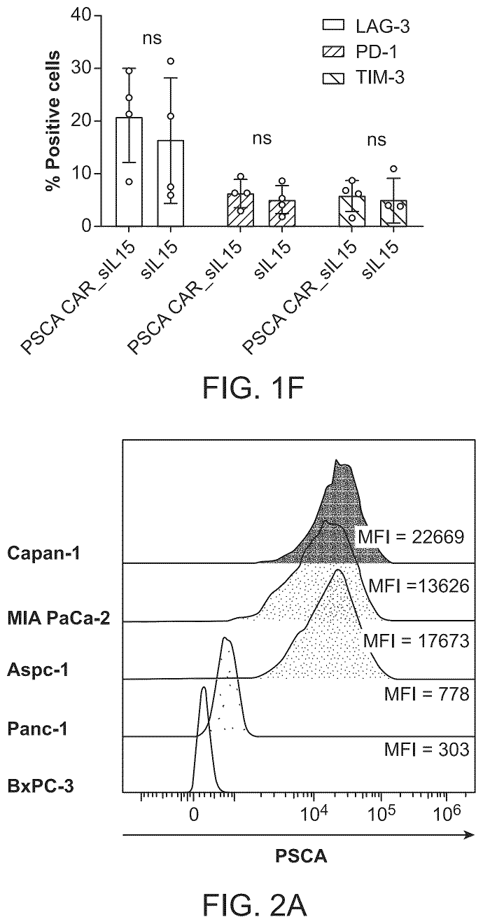

Primary NKT cells isolated and expanded from human peripheral blood mononuclear cells (PBMCs) were engineered to express soluble IL15 alone (sIL15 NKT), which can significantly enhance anti-tumor activity, or both PSCA CAR and soluble IL15 (PSCA CAR_sIL15 NKT; FIG. 1A). The constructs also carry a truncated (t)EGFR as a safety switch and a detection marker, as the cells can be depleted in vivo by administering a clinical-grade anti-EGFR antibody, cetuximab. The sIL15 NKT cells and PSCA CAR_sIL15 NKT cells show high NKT purities as around 97% cells are NKT cells (FIG. 1B). Furthermore, three days after transduction, the transduction efficiency, which are around 42%, are similar in both sIL15 NKT cells and PSCA CAR_sIL15 NKT cells by analyzing EGFR expression (FIG. 1C). We also detected the basal apoptosis levels of sIL15 and PSCA CAR_sIL15 NKT cells. Flow cytometry results showed that both sIL15 and PSCA CAR_sIL15 NKT cells are at very low-level apoptosis (FIG. 1D). Therefore, the data showed the newly generated PSCA CAR_sIL15 NKT cells are good quality with high transduction rate and high NKT cell purify. After in vitro culturing with α-galactosylceramide (α-GalCer), both sIL15 and PSCA CAR_sIL15 NKT cells could be expanded more than 5000-fold (FIG. 1E) and the expanded NKT cells expressed low levels of exhaustion markers, LAG-3, PD-1, and TIM-3 (FIG. 1F). These data demonstrated the successful engineering and manufacturing of PSCA CAR_sIL15 NKT cells.

### Example 2: PSCA CAR_sIL15 NKT Cells Exhibited Potent Anti-Tumor Activity Against PC Cell Lines In Vitro

Previously, we reported that PSCA was highly expressed in primary pancreatic tumor samples and was associated with poor patient prognosis (Teng K-Y, Mansour A G, Zhu Z, et al. Off-the-Shelf Prostate Stem Cell Antigen-Directed Chimeric Antigen Receptor Natural Killer Cell Therapy to Treat Pancreatic Cancer. Gastroenterology 2022; 162(4):1319-33). To assess the anti-tumor activity of PSCA CAR_sIL15 NKT cells in subsequent functional validation, the expression of PSCA in five different PC cell lines using flow cytometry was measured.

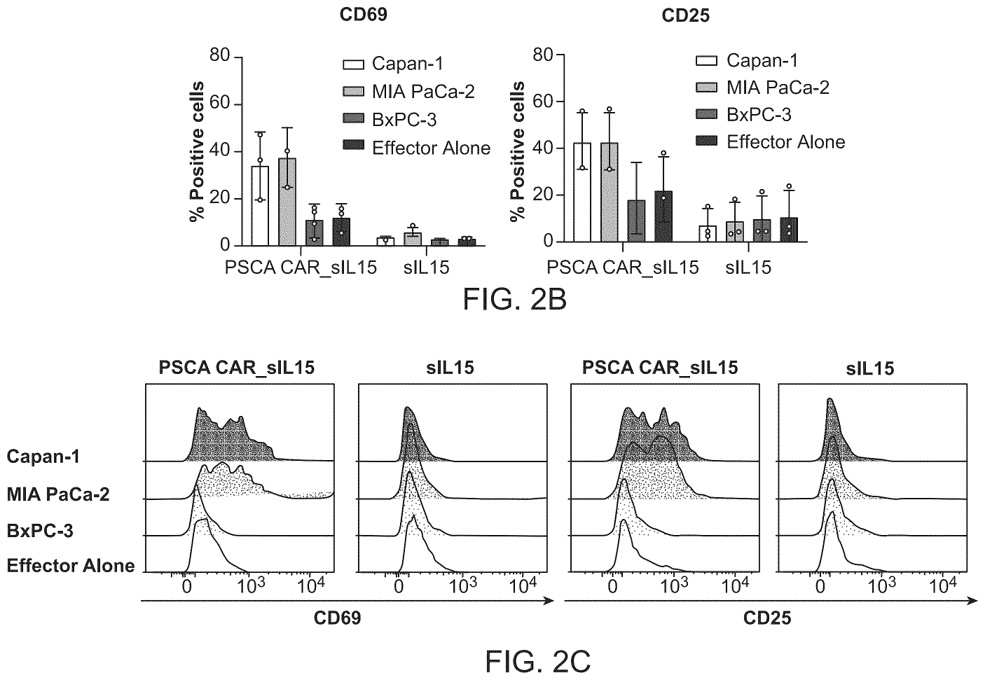

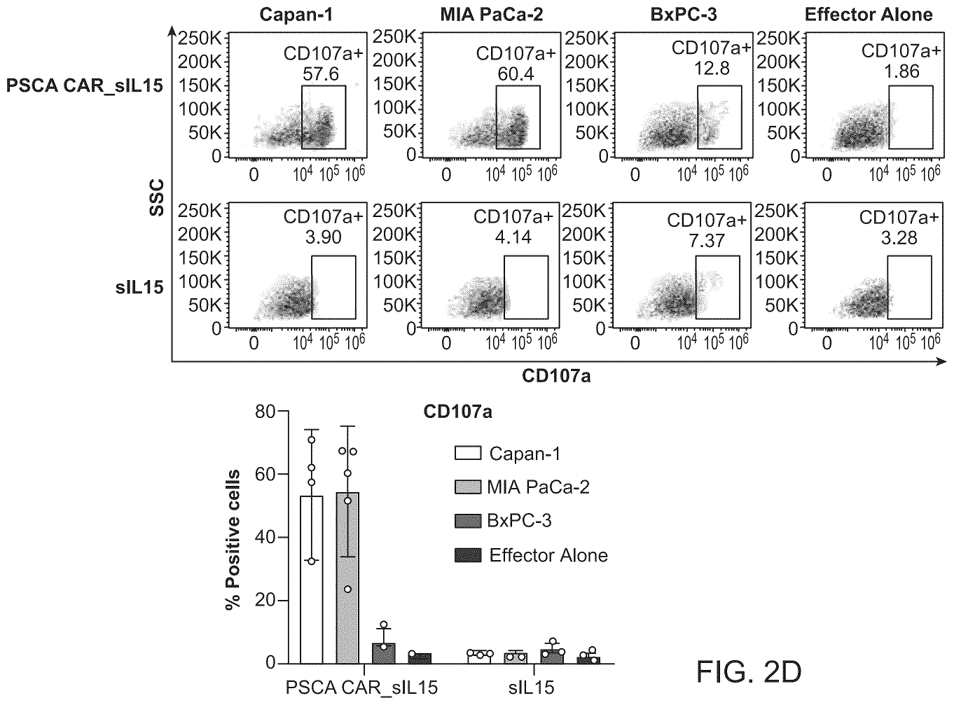

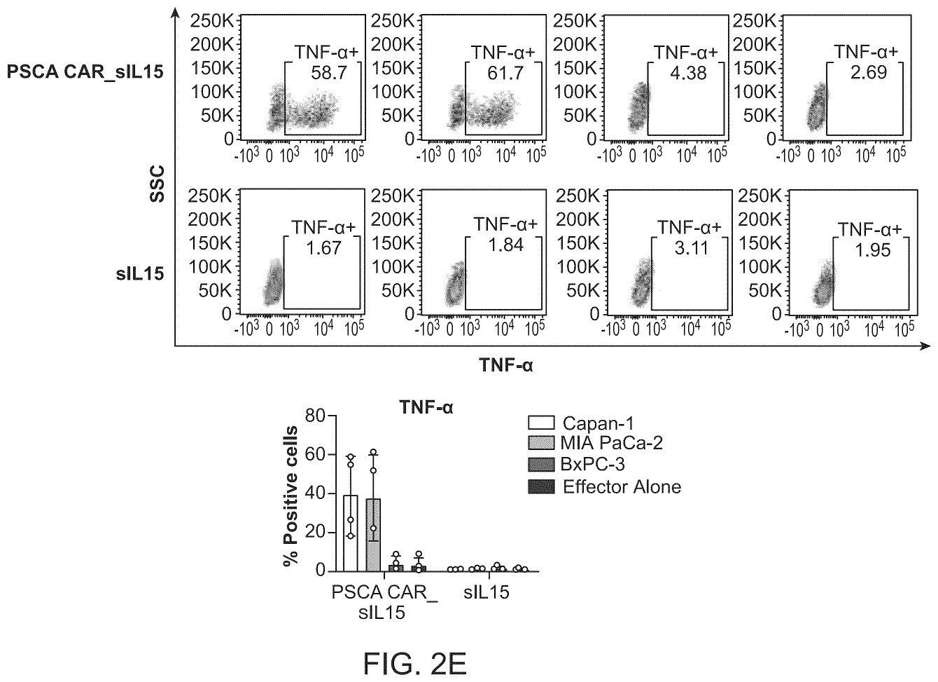

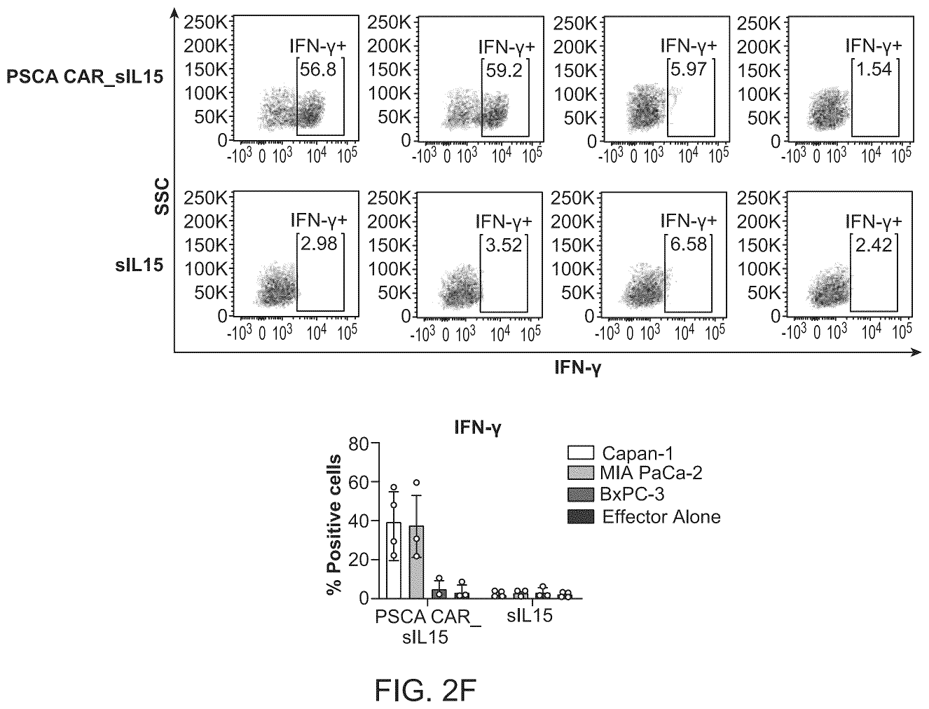

The data showed that the cell lines Capan-1, MIA Paca-2, and Aspc-1 highly express PSCA, while Panc-1 and BxPC-3 cells were PSCA-cells (FIG. 2A). To evaluate anti-PC function of PSCA CAR_sIL15 NKT cells in vitro, PSCA CAR_sIL15 NKT cells or sIL15 NKT cells were co-cultured with the different PC cells at an effector:target (E:T) ratio of 1:1 for 6 hours. Compared to sIL15 NKT cells, PSCA CAR_sIL15 NKT cells expressed significantly higher levels of the activation markers CD69 and CD25 after co-culturing with PSCA+ Capan-1 and MIA Paca-2. Furthermore, PSCA-BxPC-3 cells cannot activate either sIL15 NKT cells or PSCA CAR_sIL15 NKT cells (FIGS. 2B and 2C).

Without being bound by theory, degranulation is a prerequisite for immune cell perforase-mediated killing. PSCA CAR_sIL15 NKT cells upregulated CD107a (a surrogate marker for degranulation) expression in a PSCA-specific manner that are only induced by PSCA⁺ PC cells but not PSCA⁻ PC cells (FIG. 2D). However, both PSCA⁺ PC cells and PSCA⁻ PC cells had no effect on activating sIL15 NKT cells (FIG. 2D). Moreover, PSCA CAR_sIL15 NKT cells produced the pro-inflammatory cytokines TNF-α and IFN-γ in response to PSCA⁺ cells compared to sIL15 NKT cells (FIGS. 2E and 2F).

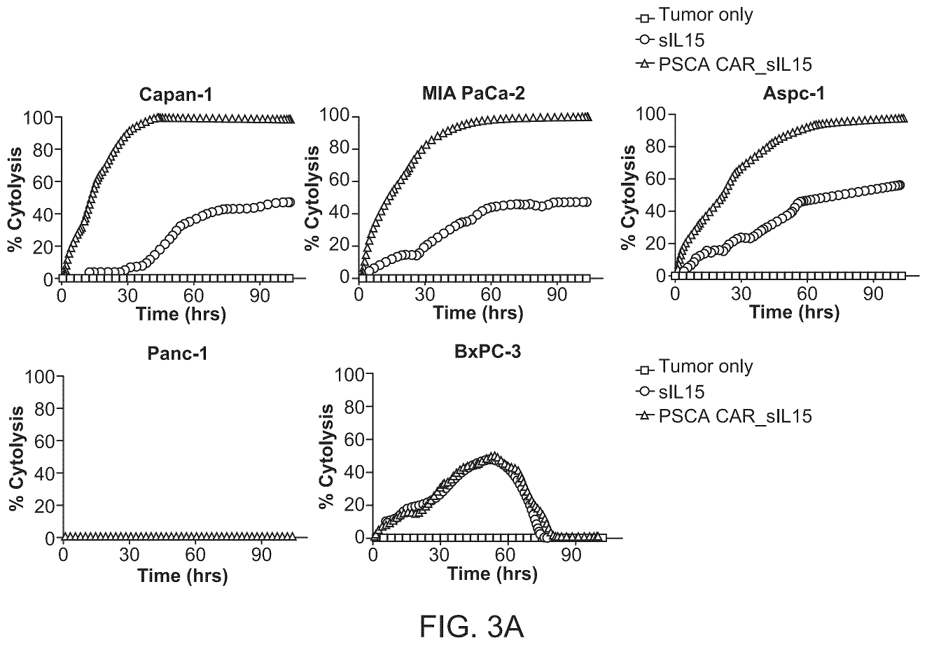

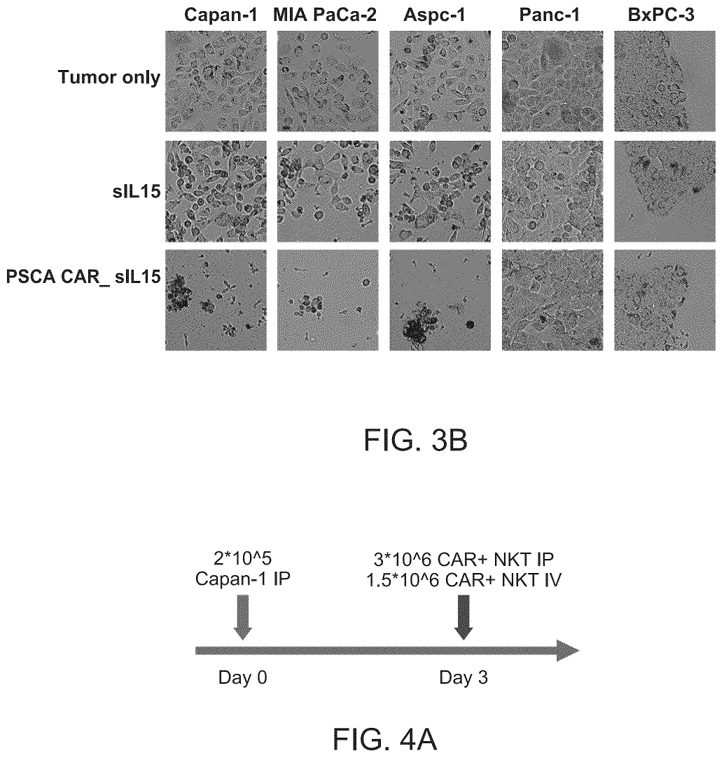

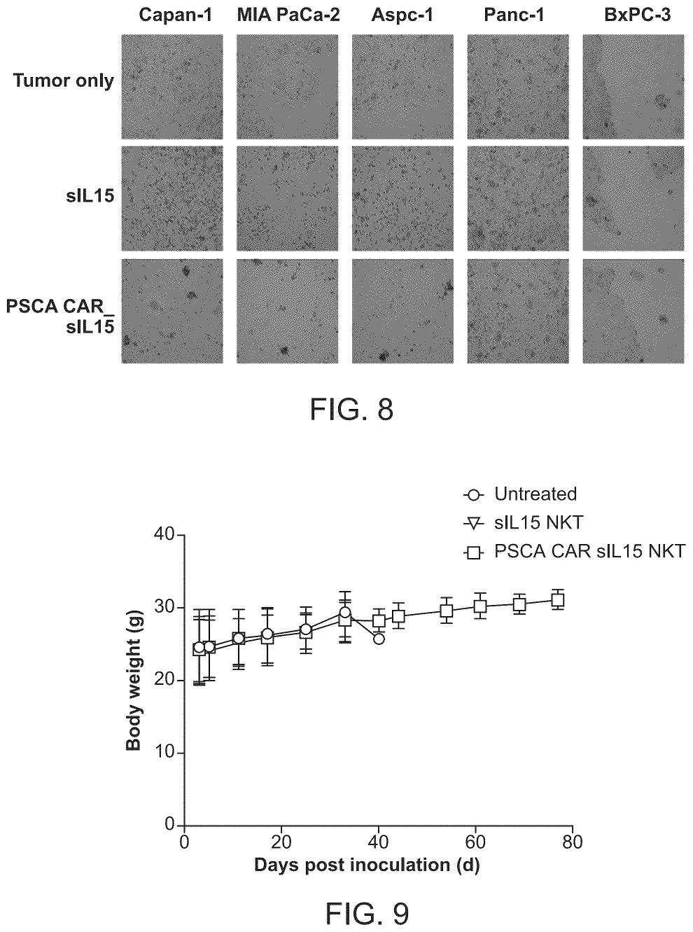

Next, the cytolytic function of PSCA CAR_sIL15 NKT cells was assessed using real time cell analysis (RTCA). PSCA CAR_sIL15 NKT cells showed robust killing against tumor cells expressing PSCA⁺ tumor cell lines Capan-1, MIA Paca-2, and Aspc-1 compared to sIL15 NKT cells, whereas both sIL15 NKT cells and PSCA CAR_sIL15 NKT cells were not cytotoxic against the PSCA-cell lines Panc-1 and BxPC-3 (FIGS. 3A and 3B, FIG. 8). Our data indicated that the cytolysis of PSCA CAR_sIL15 NKT cells was PSCA-specific. Taken together, PSCA CAR_sIL15 NKT cells exhibited potent PSCA-specific tumor-killing activity against PC cells in vitro.

### Example 3: PSCA CAR_sIL15 NKT Cells Showed Superior Therapeutic Activity in PC Metastasis Model In Vivo without Causing Significant Toxicity

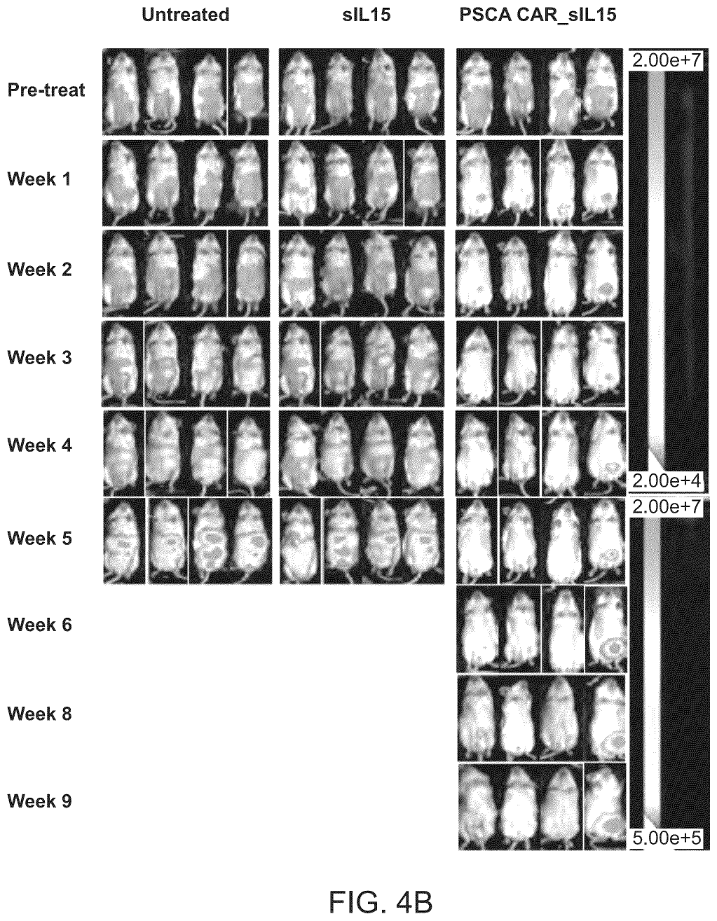

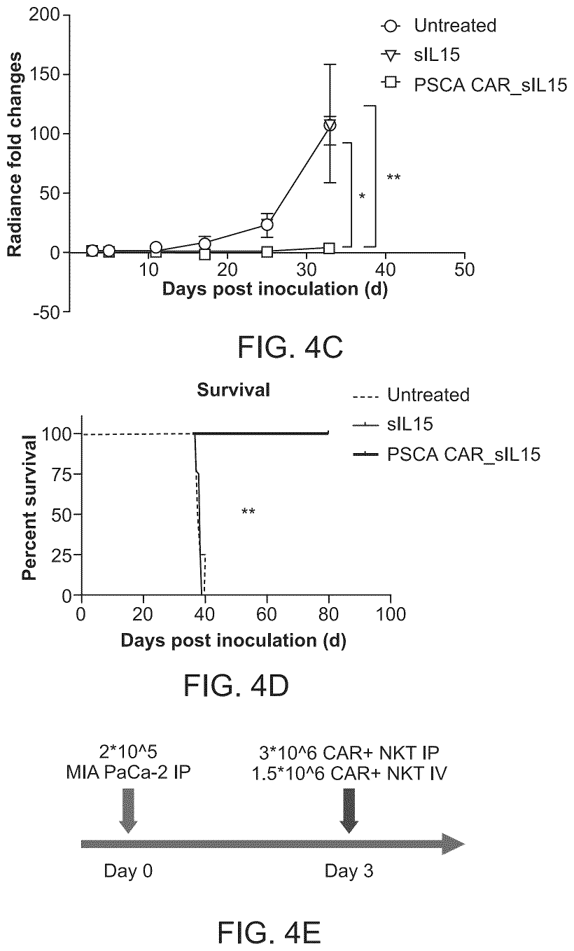

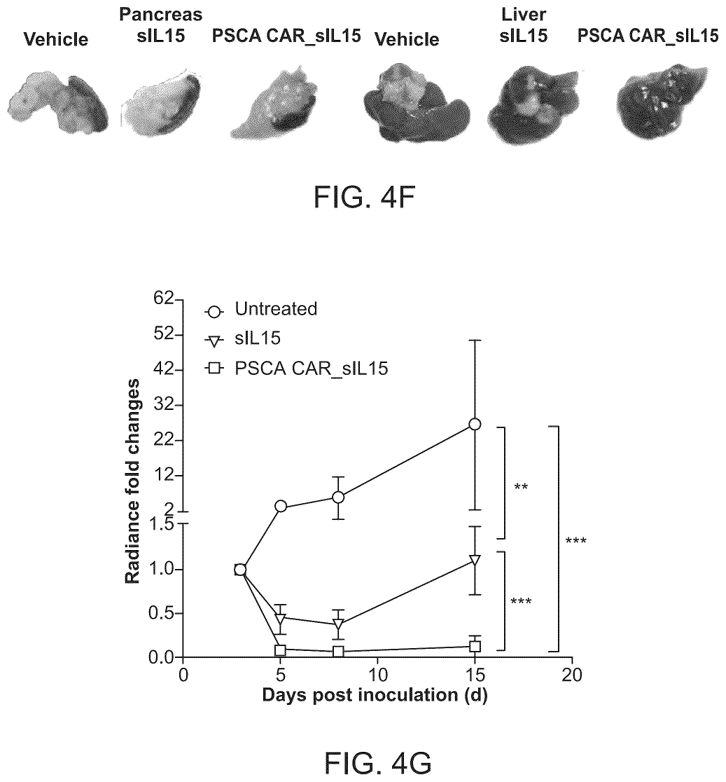

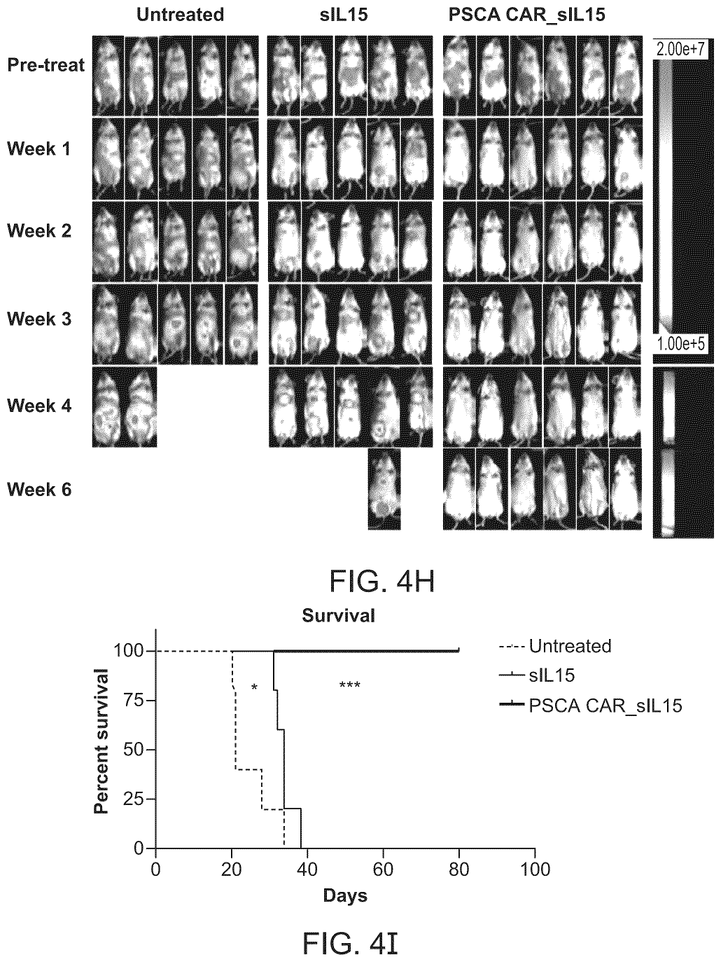

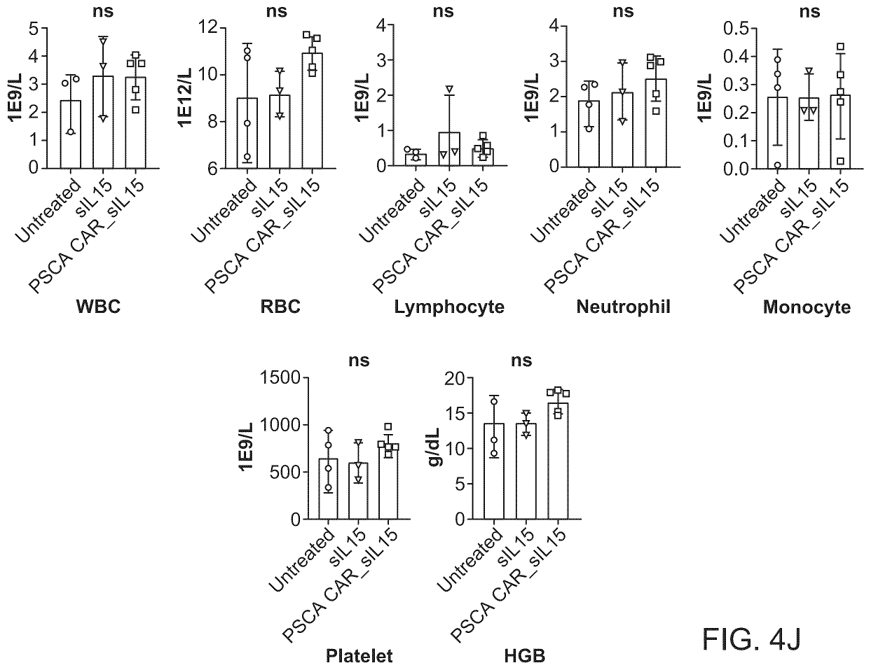

To verify the in vivo efficacy of PSCA CAR_sIL15 NKT cells, we established PC metastasis and orthotopic models. Previously, we demonstrated that a combination of intraperitoneal (IP) and intravenous (IV) injections would be beneficial in killing tumor cells in the pancreas as well as those that metastasized to the liver and lung (Teng K-Y, Mansour AG, Zhu Z, et al. Off-the-Shelf Prostate Stem Cell Antigen-Directed Chimeric Antigen Receptor Natural Killer Cell Therapy to Treat Pancreatic Cancer. Gastroenterology 2022; 162(4):1319-33). Therefore, a combination of IP and IV injections of PSCA CAR_sIL15 NKT cells was used to treat tumor-bearing mice and sIL15 NKT cells were injected as control. The process of establishing the Capan-1-based metastatic PC model and treatment is shown in FIG. 4A. Briefly, 2×10⁵ Capan-1 cells expressing firefly luciferase (FFL) gene were IP injected into NOD-scid IL2Rgamma^{null} (NSG) mice on day 0. Three days after tumor implantation, mice were treated with 3×10⁶ PSCA CAR_sIL15 NKT cells IP injected combined with 1.5×10⁶ PSCA CAR_sIL15 NKT cells IV injected. Saline and sIL15 NKT cells were treated by the same routes as control. The fluorescein-based imaging system was used to monitor the progression of tumors. Compared with the two control groups, PSCA CAR_sIL15 NKT cells significantly inhibited the progression of metastatic PC and remarkably prolonged the survival of the tumor-bearing mice (FIGS. 4B, 4C, and 4D).

We constructed another metastatic PC model using the PSCA+ cell line MIA PaCa-2 to confirm the therapeutic effects of PSCA CAR_sIL15 NKT cells (FIG. 4E). Similar therapeutic functions of PSCA CAR_sIL15 NKT cells were found that PSCA CAR_sIL15 NKT cells treatment prevented pancreatic cancer as well as the liver lesions (FIG. 4F), eradicated PC in mice, maintained complete remission and reached significantly longer survival time compared to the untreated and sIL15 NKT treated groups (FIGS. 4G, 4H, and 4I). Furthermore, hematological analysis of the blood from treated mice showed that PSCA CAR_sIL15 NKT cells treatment has no effect on blood cell counts and hemoglobin (HGB) compared to untreated group and sIL15 NKT cell group (FIG. 4J). In addition, sIL15 NKT cells also showed modest effects on delaying tumor progression in PC mice (FIGS. 4G, 4H, and 4I).

### Example 4: PSCA CAR_sIL15 NKT Cells Showed Superior Therapeutic Activity in Orthotopic PC Model In Vivo without Causing Significant Toxicity

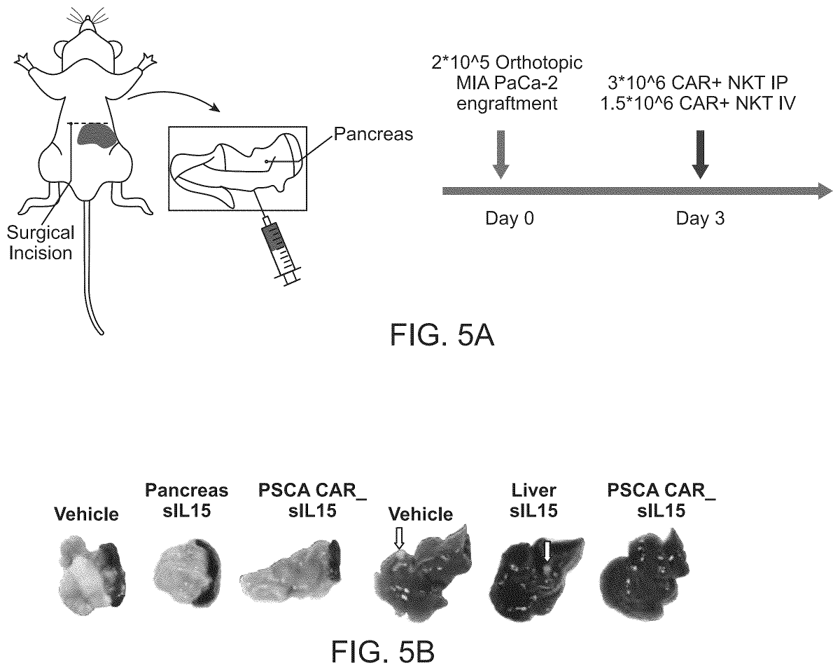

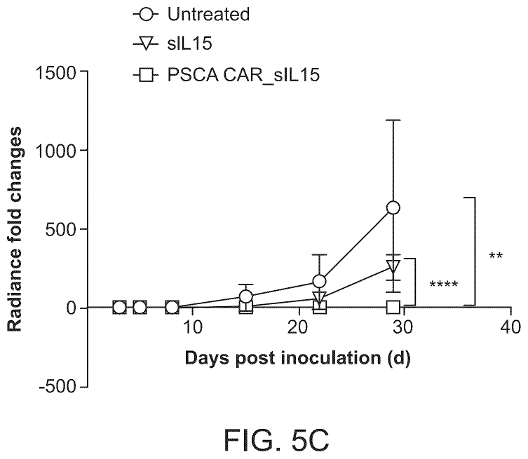

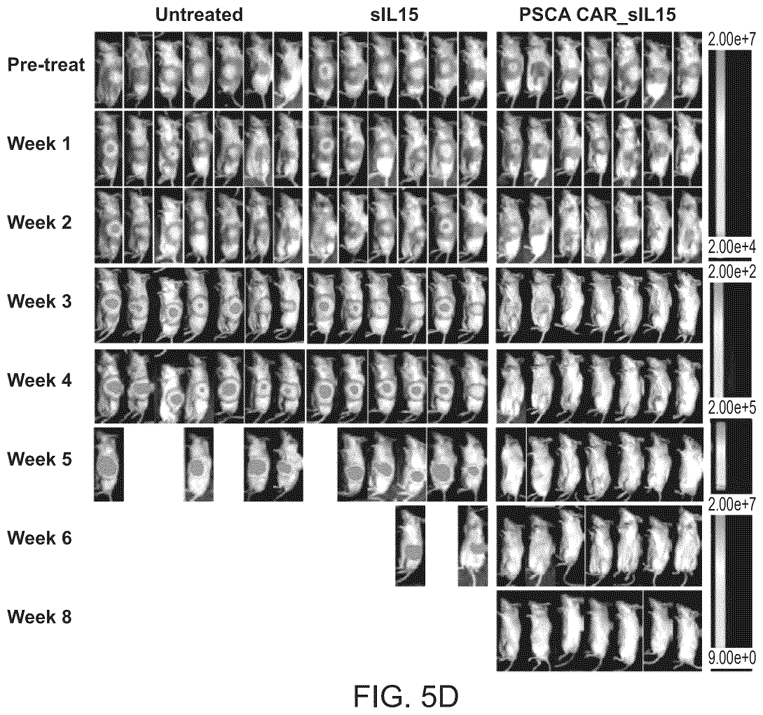

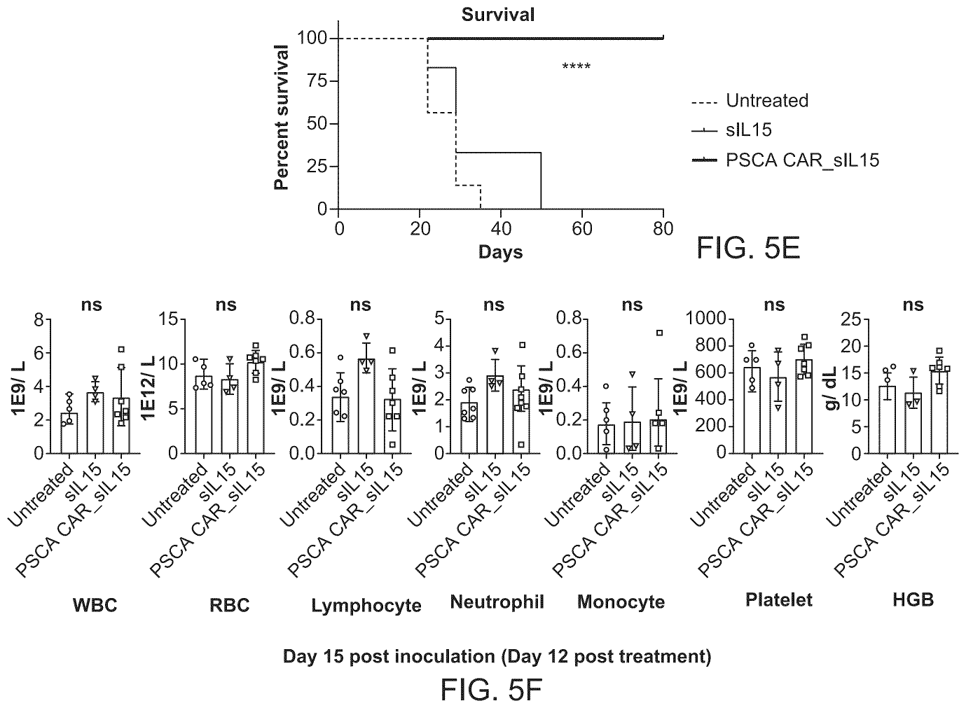

We have conducted extensive evaluation of PSCA CAR_sIL15 NKT cells in an orthotopic PC model (FIG. 5A). 2×10⁵ FFL expressing-MIA PaCa-2 cells were intrapancreatic injected on day 0. On day 3, mice were received 3×10⁶ PSCA CAR_sIL15 NKT cells IP injection and 1.5×10⁶ PSCA CAR_sIL15 NKT cells IV injection. This established model showed locoregional spread and liver metastasis, thus closely mimicking human disease. In the orthotopic PC model CAR_sIL15 NKT cells could efficiently deplete carcinoma in situ of the pancreas and reduce metastasis formation in the liver (FIG. 5B). Treatment with PSCA CAR_sIL15 NKT cells resulted in gradual clearance of orthotopic tumors and significantly prolonged survival time in mice without affecting peripheral blood counts and HGB compared to untreated and sIL15 NKT cell treatment (FIG. 5C-F).

Therefore, PSCA CAR_sIL15 NKT cells have been shown to have potent anti-tumor ability in vivo to eradicate orthotopic and metastatic tumors, as demonstrated by multiple cell lines and tumor models.

### Example 5: Gemcitabine Resistance in PC can be Overcome by PSCA CAR_sIL15 NKT Cells

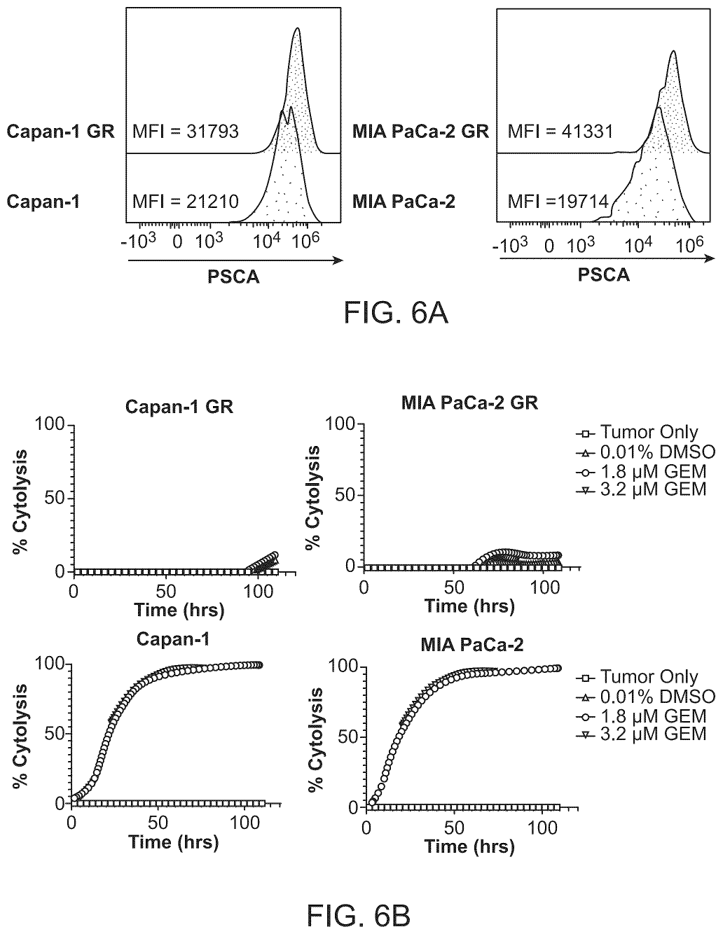

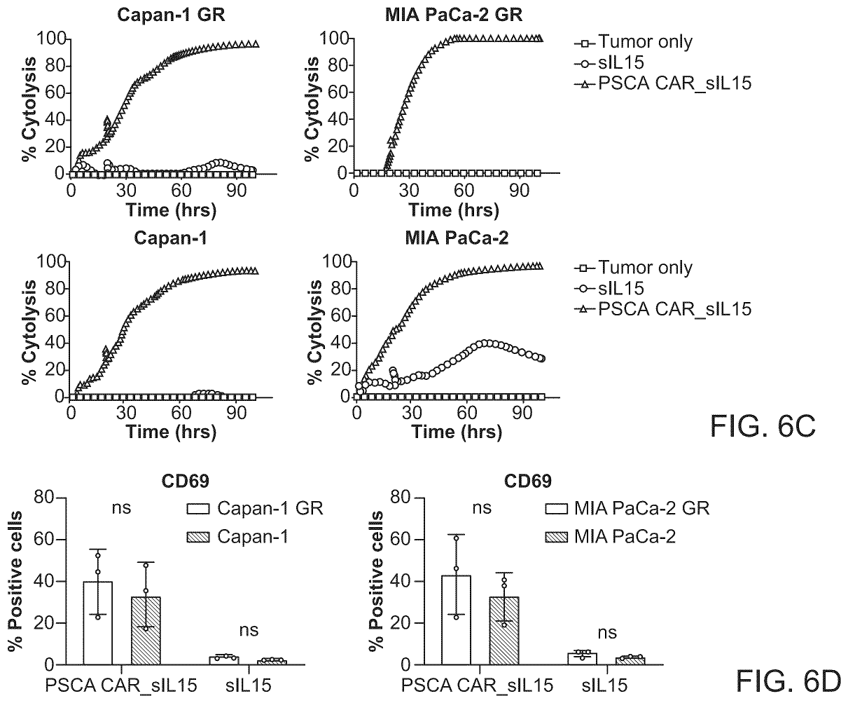

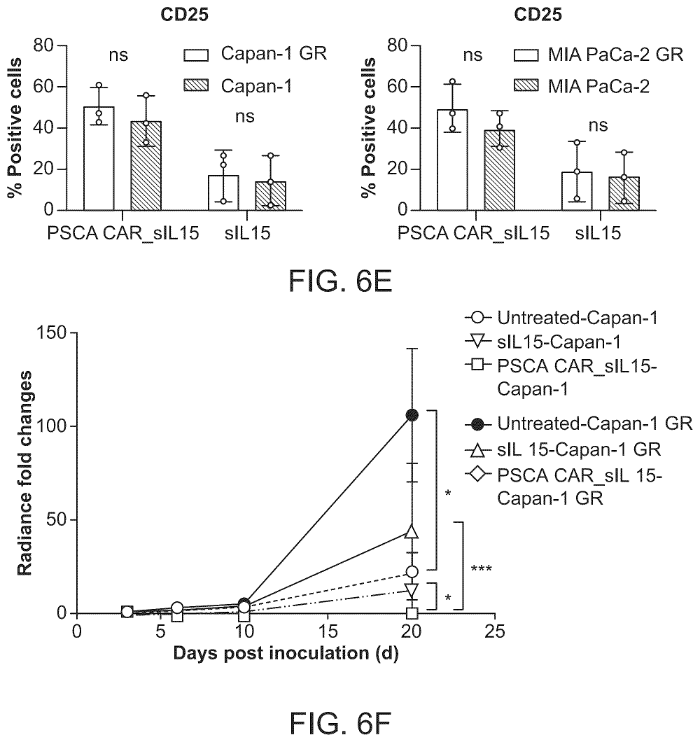

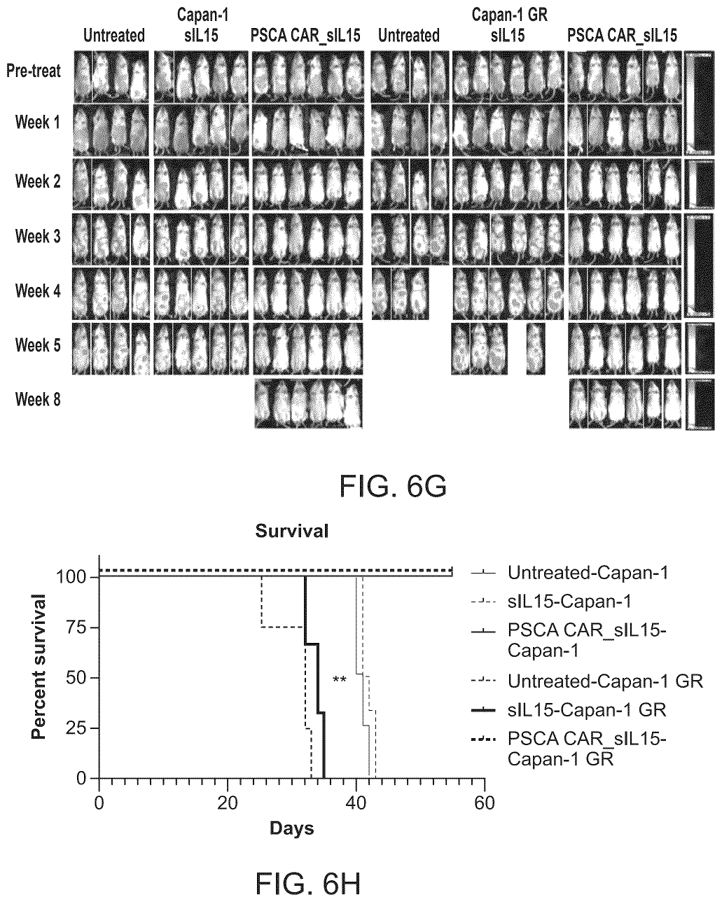

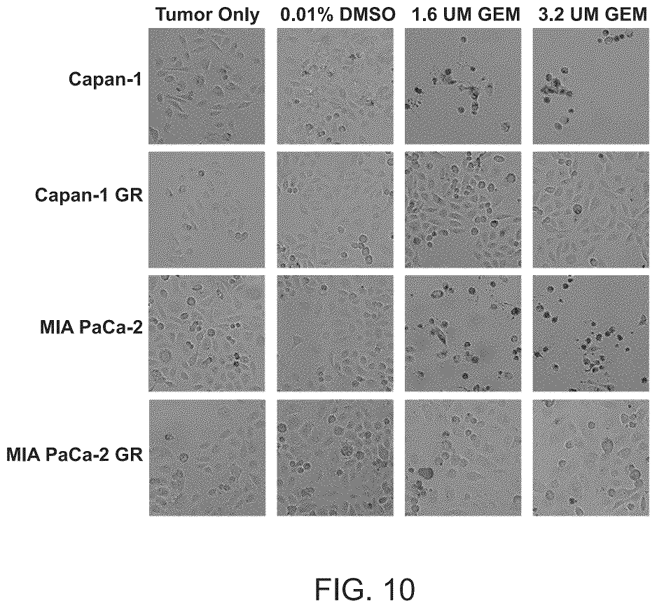

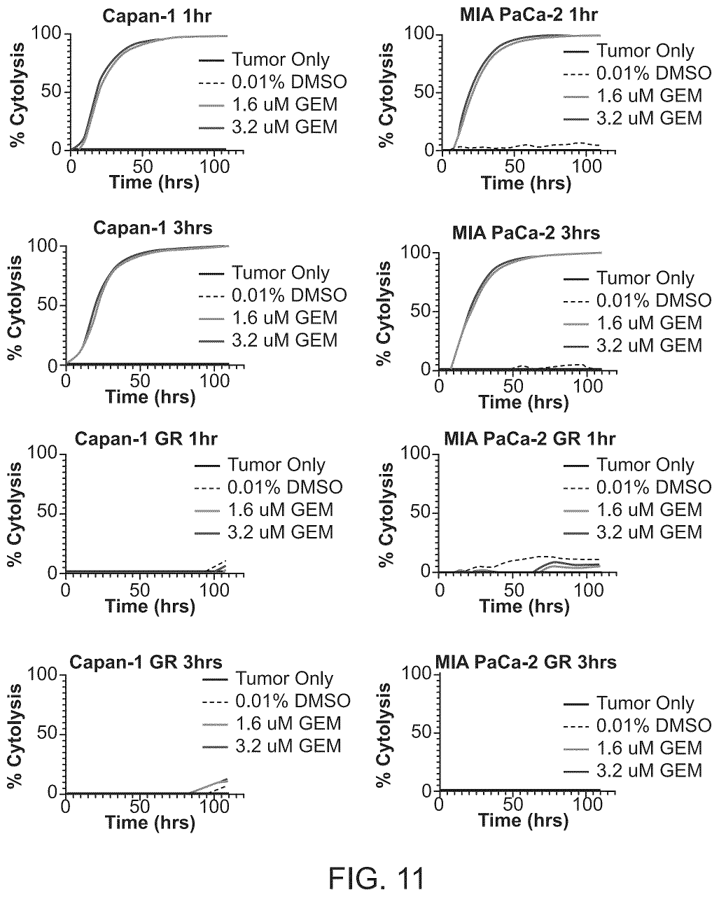

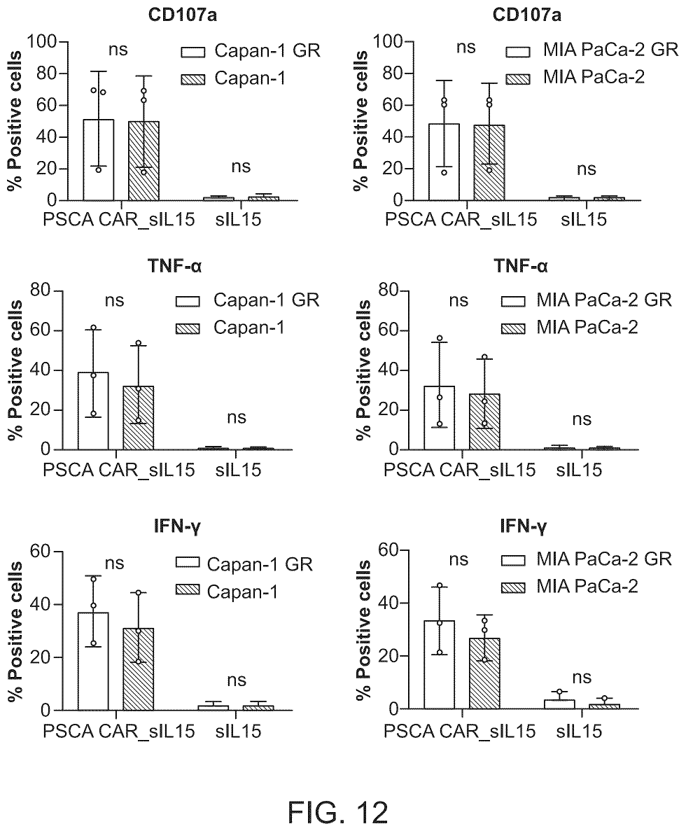

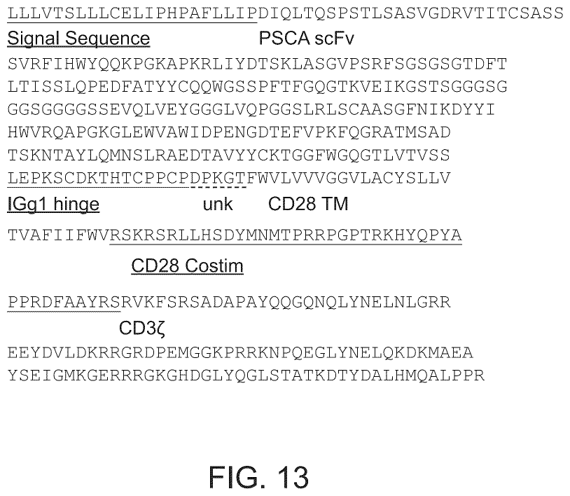

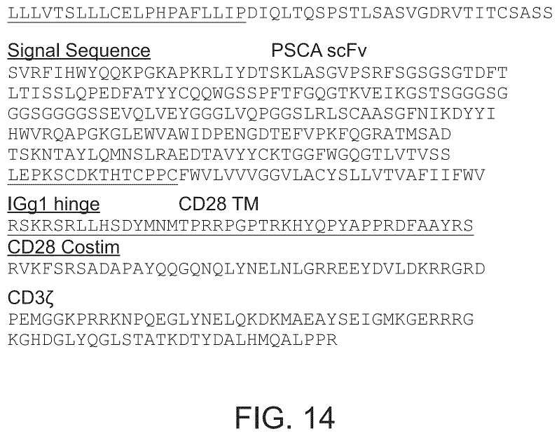

Gemcitabine-based therapy is currently the standard first-line therapy for patients with advanced PDAC. However, tumor recurrence after gemcitabine treatment may lead to short patient survival (Jia Y, Gu D, Wan J, et al. The role of GLI-SOX2 signaling axis for gemcitabine resistance in pancreatic cancer. Oncogene 2019; 38 (10): 1764-77. doi: 10.1038/s41388-018-0553-0 [published Online First: 2018 Oct. 31]). Therefore, it is critical to verify whether PSCA CAR_sIL15 NKT cells are effective in killing gemcitabine-resistant tumors in vitro and in vivo. In this case, we established two gemcitabine-resistant (GR) cell lines (Capan-1 GR and MIA Paca-2 GR) by exposing parental Capan-1 and MIA Paca-2 cells to increasing concentrations of gemcitabine for 9 months. The gemcitabine-resistant cell lines Capan-1 GR and MIA Paca-2 GR showed a slight increased expression of PSCA compared to parental Capan-1 and MIA Paca-2 cells (FIG. 6A). Compared to the parental cell lines, Capan-1 GR and MIA Paca-2 GR were not killed by gemcitabine at concentrations of 1.6 uM and 3.2 uM, either without medium change or with medium change at 1 h or 3 h (FIG. 6B, FIGS. 10 and 11). We explored the anti-PC effect of PSCA CAR_sIL15 NKT cells targeting Capan-1 GR and MIA Paca-2 GR cells in vitro. RTCA results indicated that PSCA CAR_sIL15 NKT cells still maintained potent killing function against Capan-1 GR and MIA Paca-2 GR compared to parental cells. We also measured the levels of CD25, CD69, CD107a, TNF-α, and IFN-γ expression of PSCA CAR_sIL15 NKT cells after co-incubation with GR cells and the parental cells. There were comparable levels of CD25, CD69, CD107a, TNF-α, and IFN-γ expression when PSCA CAR_sIL15 NKT cells targeting GR cells and the parental cells (FIGS. 6C-6E, FIG. 12). Next, we evaluated the therapeutic effect of PSCA CAR_sIL15 NKT cells against Capan-1 GR cells in vivo. The parental Capan-1 cells were injected as control. Compared to parental Capan-1 cells, Capan-1 GR cells was found to have a more rapid progression with shorter survival time in vivo even in the absence of gemcitabine (FIGS. 6F-6H). In either Capan-1 GR or Capan-1 established mouse tumor models, a single dose of PSCA CAR_sIL15 NKT cell treatment significantly suppressed tumor progression compared to sIL15 NKT cells (FIGS. 6F-6H). Taken together, PSCA CAR_sIL15 NKT cells exhibit superior in vivo and in vitro killing activity against gemcitabine-resistant PC cells, which have more aggressive properties.

### Example 5: Off-the-Shelf PSCA CAR_sIL15 NKT Cells Demonstrated Excellent Antitumor Function without Causing GvHD

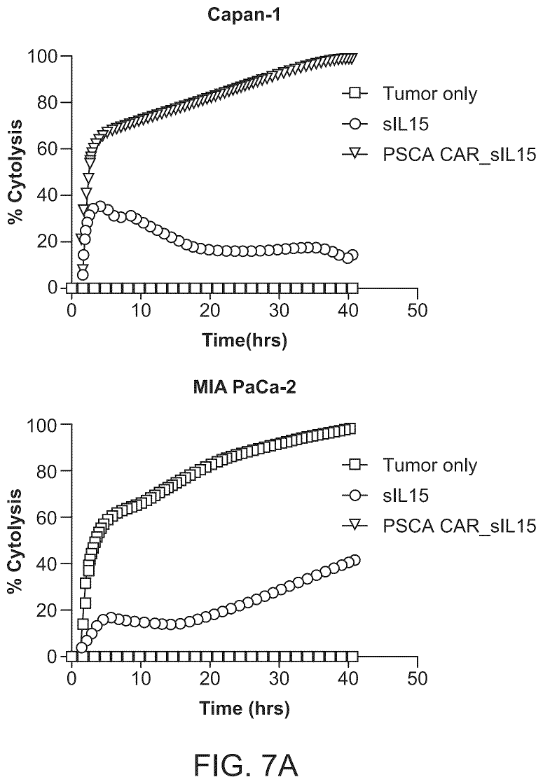

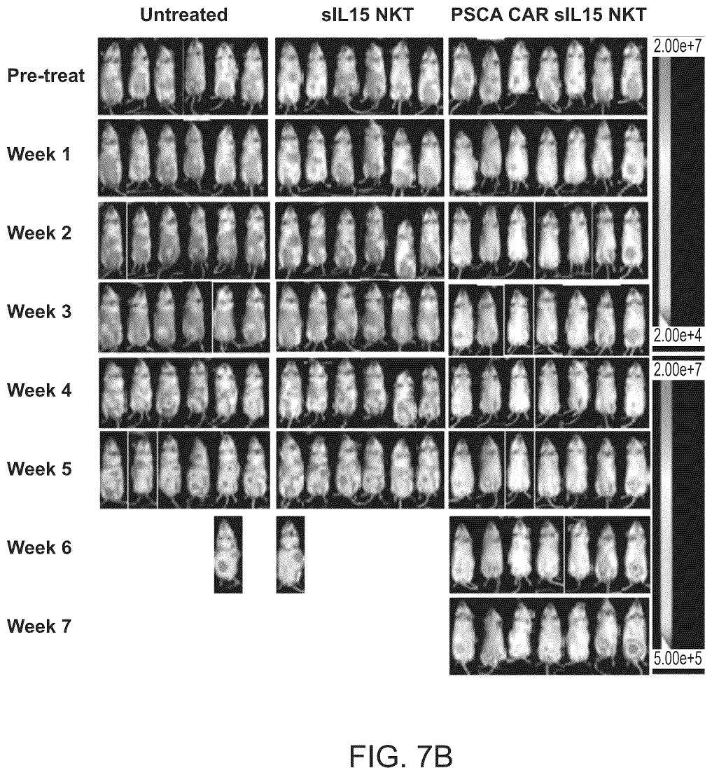

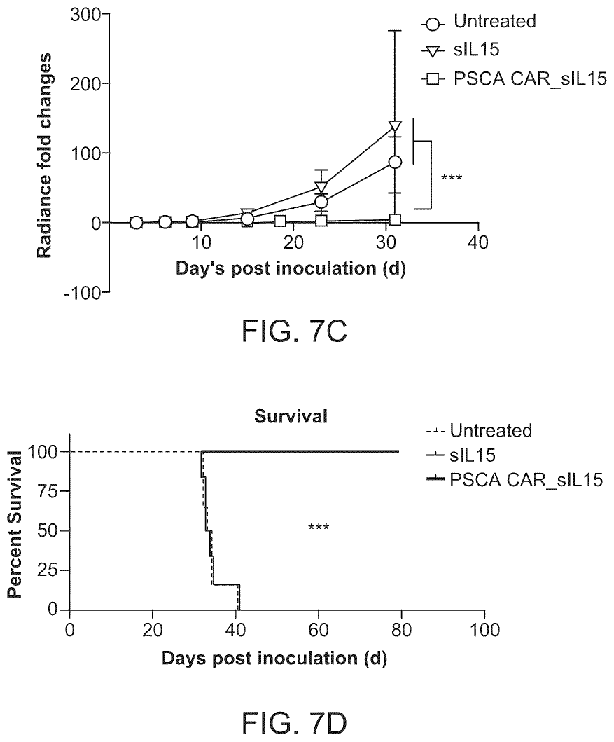

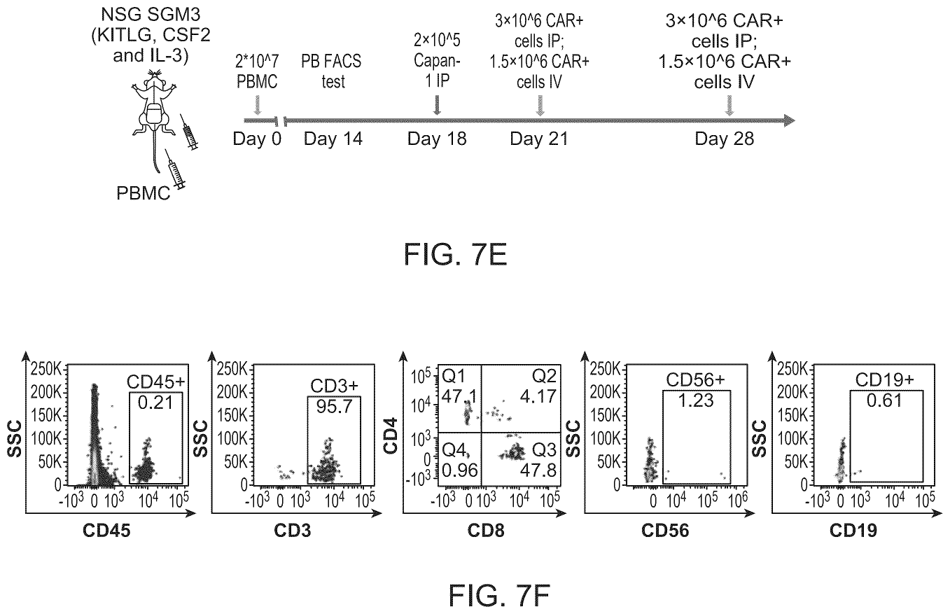

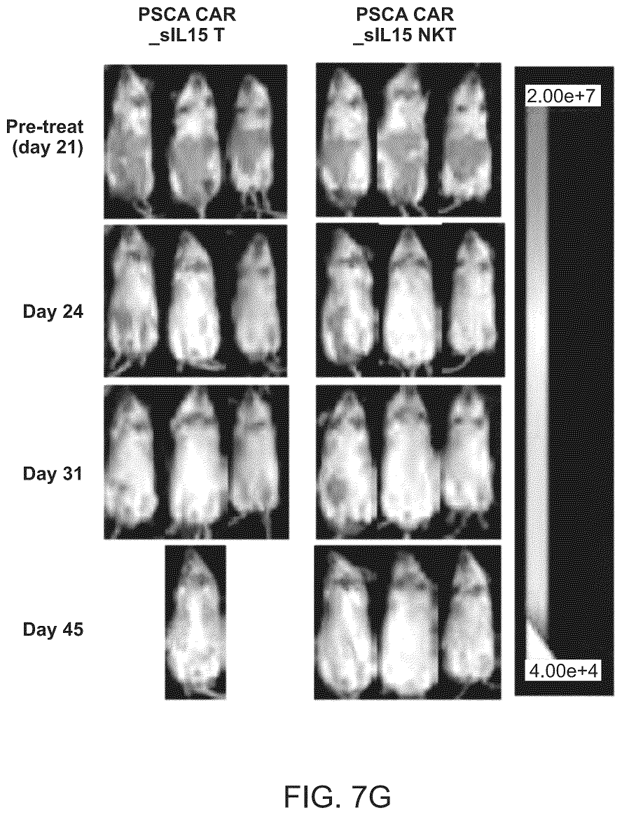

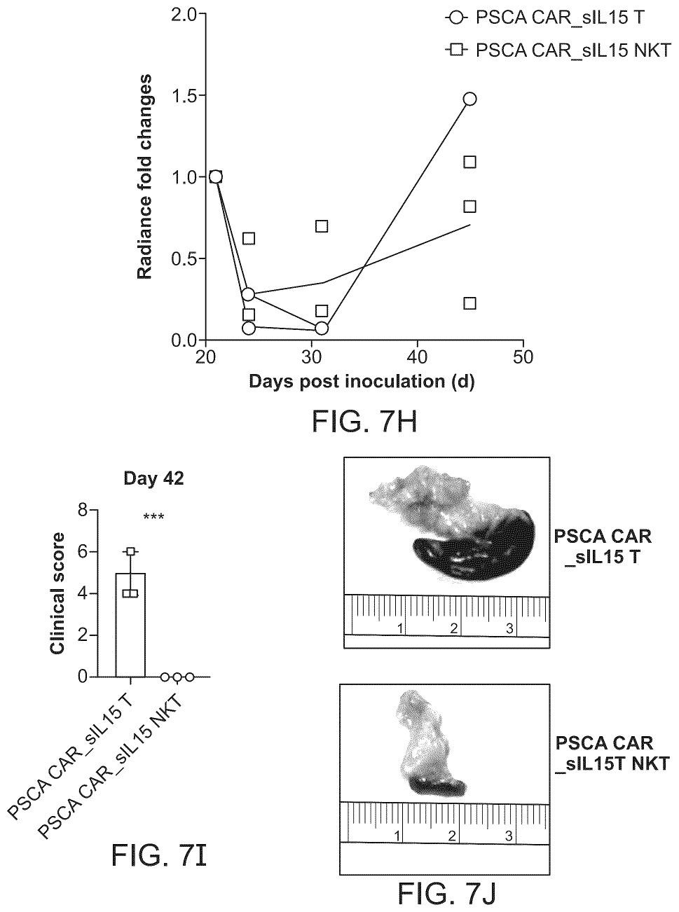

Off-the-shelf CAR-based products significantly shorten the production time and reduce the cost of CAR-based cell therapies. To this end, we validated the anti-tumor effects and safety of off-the-shelf PSCA CAR_sIL15 NKT cells in vitro and in vivo, including the risk of developing GvHD. PSCA CAR_sIL15 NKT cells recovered from cryopreservation still displayed robust killing ability against Capan-1 and MIA Paca-2 measured by RTCA (FIG. 7A). We then examined the anti-tumor activity of off-the-shelf PSCA CAR_sIL15 NKT cells in vivo using Capan-1 established metastatic PC model, and the results showed that off-the-shelf PSCA CAR_sIL15 NKT cells still showed superior therapeutic efficacy in inhibiting the progression of metastatic PC and prolonged the survival time of mice (FIGS. 7B-7D). To investigate the risk of off-the-shelf PSCA CAR_sIL15 NKT cell therapy leading to GvHD compared to PSCA CAR_sIL15 T cell therapy, NSG SGM3 mice received 2×10⁷ PBMC via the tail vein for establishing humanized mice on day 0 (FIG. 7E). Repopulated human T lymphocytes, including CD3⁺ CD4⁺ and CD3⁺ CD8⁺ cells, were observed in the peripheral blood of mice 14 days after transplantation, while few CD19⁺ or CD56⁺ cells were detected (FIG. 7F), indicating the successful establishment of humanized immune system. The humanized mice were intraperitoneally inoculated with 2×10⁵ Capan-1 on day 18, and allogeneic PSCA CAR_sIL15 NKT cells or PSCA CAR_sIL15 T cells (3×10⁶ CAR⁺ cells IP, 1.5×10⁶ CAR⁺ cells IV; the same donor-derived NKT/T cells) were administered twice on days 21 and 28, respectively (FIG. 7E). Tumor progression was monitored by luciferase-based imaging and the results showed that there was no significant difference of anti-PC ability between PSCA CAR_sIL15 NKT cells and PSCA CAR_sIL15 T cells, as both the cells significantly suppressed tumor progression in mice (FIGS. 7G-7H). Clinical GvHD score were monitored by evaluating systemic symptoms including weight loss, posture, activity, fur texture, and skin integrity. On day 42 PSCA CAR_sIL15 T cells treatment showed significantly higher GvHD scores compared to PSCA CAR_sIL15 NKT cells treatment (FIG. 7I). Tumor-bearing mice treated with PSCA CAR_sIL15 T cells developed giant spleens, while PSCA CAR_sIL15 NKT cells-treated mice had normal spleen size (FIG. 7J). Therefore, in contrast to PSCA CAR_sIL15 T cells that cause lethal GvHD in humanized NSG mice, NKT cells expressing the same CAR construct exhibited similar efficacy but with a lower toxicity profile.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A population of cells comprising NK T cells (NKT cells) harboring:
(a) a nucleic acid molecule encoding both: a chimeric antigen receptor (CAR), the CAR comprising: a single chain variable domain (scFv) targeting prostate stem cell antigen (PSCA), a spacer, a transmembrane domain, a co-stimulatory domain, and a CD3ζ signaling domain; and polypeptide comprising a human IL-15 domain that binds to IL-15 receptor; or
(b) a first nucleic acid molecule encoding a chimeric antigen receptor (CAR), the CAR comprising: a single chain variable domain (scFv) targeting prostate stem cell antigen (PSCA), a spacer, a transmembrane domain, a co-stimulatory domain, and a CD3ζ signaling domain and second nucleic acid molecule encoding a polypeptide comprising a human IL-15 domain that binds to IL-15 receptor;
wherein the scFv comprises a variable heavy chain (HC) and a variable light chain (LC),
wherein the HC comprises complementarity-determining regions: CDRH1 comprising the amino acid sequence of SEQ ID NO: 50, CDRH2 comprising the amino acid sequence of SEQ ID NO: 51, and CDRH3 comprising the amino acid sequence of GGF, and
wherein the LC comprises complementarity-determining regions: CDRL1 comprising the amino acid sequence of SEQ ID NO: 53, CDRL2 comprising the amino acid sequence of SEQ ID NO: 54, and CDRL3 comprising the amino acid sequence of SEQ ID NO: 55.

2.-3. (canceled)

4. The population of cells of claim 1, wherein the scFv comprises the amino acid sequences of SEQ ID NOs: 32 and 33.

5. The population of cells of claim 1, wherein the scFv comprises or consists of the amino acid sequence of SEQ ID NOs: 1, 40, or a variant thereof or each having up to 5, 4, 3, 2 or 1 amino acid substitutions, wherein the amino acid substitutions are conservative and not in the CDRs.

6. The population of cells of claim 1, wherein:
(a) the spacer comprises or consists of the amino acid sequence of any of SEQ ID NOs: 3-12 and 28;
(b) the transmembrane domain comprises or consists of the amino acid sequence of any of SEQ ID NOs: 13-20 and 34;
(c) costimulatory domain comprises or consists of the amino acid sequence of any of SEQ ID NOs: 22-26;
(d) the spacer comprises or consists of the amino acid sequence of any of SEQ ID NOs: 3-12 and 28; the transmembrane domain comprises or consists of the amino acid sequence of any of SEQ ID NOs: 13-20 and 34; costimulatory domain comprises or consists of the amino acid sequence of any of SEQ ID NOs: 22-26; and the CD3ζ signaling domain comprises or consists of the amino acid sequence of SEQ ID NO: 21 or a variant thereof selected from SEQ ID NOs: 56-62;
(e) the spacer comprises the amino acid sequence of SEQ ID NO: 28, the transmembrane domain comprises the amino acid sequence of SEQ ID NO: 14 or 15, the co-stimulatory domain comprises the amino acid sequence of SEQ ID NO: 22 or 23, and the CD3ζ domain comprises the amino acid sequence of SEQ ID NO: 2; or
(f) the spacer comprises the amino acid sequence of SEQ ID NO: 28, the transmembrane domain comprises the amino acid sequence of SEQ ID NO: 14, the co-stimulatory domain comprises the amino acid sequence of SEQ ID NO: 22, and the CD3ζ domain comprises the amino acid sequence of SEQ ID NO: 21.

7. The population of cells of claim 1, wherein the CAR comprises the amino acid sequence of any one of SEQ ID NOs: 40-45 & 69.

8. The population of cells of claim 1, wherein at least 60%, 70%, 80%, 85%, 90% or 95% of the cells are NKT cells.

9. The population of cells of claim 1, wherein at least 30%, 35%, 40%, 45%, 50%, 55%, 60%, 65% or 75% of cells harbor a nucleic acid molecule encoding the CAR.

10. The population of cells of claim 1, wherein at least 30%, 35%, 40%, 45%, 50%, 55%, 60%, 65% or 75% of NKT cells harbor a nucleic acid molecule encoding the CAR.

11.-12. (canceled)

13. The population of cell of claim 1, wherein the human IL-15 domain comprises or consists of the amino acid sequence of any of SEQ ID NOs: 63-65.

14. The population of cells of claim 1, wherein the polypeptide comprising an IL-15 domain comprises a signal sequence for directing secretion of the IL-domain.

15. The population of cells of claim 1, wherein the PSCA CAR and the IL-15 domain are co-expressed.

16. The population of cells of claim 1, wherein the cells comprise a nucleic acid molecule encoding a PSCA CAR followed by a skip sequence, a signal sequence and an IL-15 domain wherein the PSCA CAR includes a signal sequence.

17. The population of cells of claim 1, wherein the cells comprise natural killer cells (NK cells), natural killer T cells (NKT cells) or both.

18. A method for treating a cancer or a solid tumor that expresses PSCA in a human subject in need thereof, comprising administering the subject a therapeutically effective composition comprising the population of cells of claim 17.

19. (canceled)

20. A method of reducing PSCA-positive cells in a human subject, comprising administering the subject a composition comprising the population of cells of claim 17.

21.-22. (canceled)

23. The method of claim 18, wherein the solid tumor or cancer is any one or more of: a gall bladder cancer, a gall bladder adenocarcinoma, a pancreatic cancer, a PSCA-expressing NSCLC, a prostate cancer, a urinary bladder cancer, a cervical cancer, an esophageal cancer, or a gastric cancer.

24. The method of claim 18, wherein the population composition is administered locally or systemically.

25. The method of claim 18, wherein the cell or population is administered by single or repeat dosing.

26.-32. (canceled)


SEQUENCE LISTING (79 sequences)

SEQ ID NO: 1 - protein, 236 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 2 - protein, 10 aa, synthetic construct
GGGSSGGGSG

SEQ ID NO: 3 - protein, 12 aa, synthetic construct
ESKYGPPCPPCP

SEQ ID NO: 4 - protein, 12 aa, synthetic construct
ESKYGPPCPSCP

SEQ ID NO: 5 - protein, 22 aa, synthetic construct
ESKYGPPCPPCPGGGSSGGGSG

SEQ ID NO: 6 - protein, 39 aa, synthetic construct
IEVMYPPPYLDNEKSNGTIIHVKGKHLCPSPLFPGPSKP

SEQ ID NO: 7 - protein, 48 aa, synthetic construct
AKPTTTPAPRPPTPAPTIASQPLSLRPEACRPAAGGAVHTRGLDFACD

SEQ ID NO: 8 - protein, 45 aa, synthetic construct
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 27 - protein, 24 aa, synthetic construct
LEGGGEGRGSLLTCGDVEENPGPR

SEQ ID NO: 28 - protein, 16 aa, synthetic construct
LEPKSCDKTHTCPPCP

SEQ ID NO: 29 - protein, 20 aa, synthetic construct
METDTLLLWVLLLWVPGSTG

SEQ ID NO: 30 - protein, 18 aa, synthetic construct
MGWSSIILFLVATATGVH

SEQ ID NO: 31 - protein, 20 aa, synthetic construct
MYRMQLLSCIALSLALVTNS

SEQ ID NO: 32 - protein, 109 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGST

SEQ ID NO: 33 - protein, 112 aa, synthetic construct
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 34 - protein, 21 aa, Homo sapiens
PFFFCCFIAVAMGIRFIIMVA

SEQ ID NO: 35 - protein, 19 aa, synthetic construct
MDWIWRILFLVGAATGAHS

SEQ ID NO: 36 - protein, 22 aa, Homo sapiens
MLLLVTSLLLCELPHPAFLLIP

SEQ ID NO: 37 - protein, 15 aa, synthetic construct
GGGGSGGGGSGGGGS

SEQ ID NO: 38 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 39 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 40 - protein, 236 aa, synthetic construct
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGGGSGGGSGGGGSSDIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTS

SEQ ID NO: 41 - protein, 458 aa, synthetic construct
LLLVTSLLLCELPHPAFLLIPDIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 437 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 453 aa, synthetic construct
LLLVTSLLLCELPHPAFLLIPDIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 432 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSLEPKSCDKTHTCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 452 aa, synthetic construct
LLLVTSLLLCELPHPAFLLIPDIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSLEPKSCDKTHTCPPCFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 18 aa, synthetic construct
EGRGSLLTCGDVEENPGP

SEQ ID NO: 47 - protein, 22 aa, synthetic construct
GSGATNFSLLKQAGDVEENPGP

SEQ ID NO: 48 - protein, 16 aa, synthetic construct
SSGGGSGGGSGGGGSS

SEQ ID NO: 49 - protein, 15 aa, synthetic construct
SGGGSGGGSGGGGSS

SEQ ID NO: 50 - protein, 5 aa, synthetic construct
DYYIH

SEQ ID NO: 51 - protein, 17 aa, synthetic construct
WIDPENGDTEFVPKFQG

SEQ ID NO: 52 - skipped

SEQ ID NO: 53 - protein, 10 aa, synthetic construct
SASSSVRFIH

SEQ ID NO: 54 - protein, 7 aa, synthetic construct
DTSKLAS

SEQ ID NO: 55 - protein, 9 aa, synthetic construct
QQWGSSPFT

SEQ ID NO: 56 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 57 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 58 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 59 - protein, 112 aa, synthetic construct
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 60 - protein, 112 aa, synthetic construct
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 61 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 62 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 63 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 64 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 65 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - protein, 23 aa, Homo sapiens
VAISTSTVLLCGLSAVSLLACYL

SEQ ID NO: 67 - protein, 4 aa, synthetic construct
DYYI

SEQ ID NO: 68 - protein, 5 aa, synthetic construct
DPKGY

SEQ ID NO: 69 - protein, 431 aa, synthetic construct
DIQLTQSPSTLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSLEPKSCDKTHTCPPCFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70-78 - skipped (9 entries)

SEQ ID NO: 79 - protein, 129 aa, synthetic construct; identical to SEQ ID NO: 9